(19) United States
(12) Patent Application Publication
(10) Pub. No.: US 2026/0308524 A1
(43) Pub. Date: Oct. 8, 2026

(54) COMPOSITIONS COMPRISING AND METHODS OF USING DENDRITIC CELL EXOSOMES

(71) Applicant: BAYLOR COLLEGE OF MEDICINE, Houston, TX (US)

(72) Inventors: Willliam K. DECKER, Houston, TX (US); Damilola OYEWOLE-SAID, Houston, TX (US); Vanaja KONDURI, Houston, TX (US); Nalini BISHT, Houston, TX (US); Keenan ERNSTE, Houston, TX (US); Matthew M. HALPERT, Houston, TX (US)

(73) Assignee: BAYLOR COLLEGE OF MEDICINE, Houston, TX (US)

(21) Appl. No.: 19/483,043

(22) PCT Filed: May 10, 2024
(86) PCT No.: PCT/US2024/028938

§ 371 Date: Nov. 10, 2025

Related U.S. Application Data
(60) Provisional application No. 63/501,501, filed on May 11, 2023.

(51) Int. Cl. C12N 5/0784; A61K 35/15; A61K 38/00; A61P 35/00; C07K 16/28; C12N 5/0783

(52) U.S. Cl. CPC C12N 5/0639; A61K 35/15; A61K 38/00; A61P 35/00; C07K 16/2818; C12N 5/0636; C12N 2501/2312; C12N 2501/998

(57) ABSTRACT

Provided herein are methods to generate extracellular vesicles (EV) from large cultures of polarized DC as well as additional procedures to separate CTLA-4⁺ and CTLA-4^{neg} EV from each other and recover both populations of EV in a state that is physically and functionally intact, and as well as compositions comprising the EV obtained by the methods. Also provided are the use of CTLA-4^{neg} EV for the treatment of cancer, and the use of CTLA-4⁺ EV for the treatment of GVHD and other T-cell mediated autoimmune conditions.

### REFERENCE TO RELATED APPLICATIONS

The present application claims the priority benefit of U.S. provisional application No. 63/501,501, filed May 11, 2023, the entire contents of which are incorporated herein by reference.

### STATEMENT OF FEDERALLY SPONSORED RESEARCH

This invention was made with government support under Grant No. AI127387 and Grant No. AI153326 awarded by the National Institutes of Health. The government has certain rights in the invention.

### BACKGROUND

### 1. Field

The present invention relates generally to the fields of immunology and medicine. More particularly, it concerns compositions comprising, methods of making, and methods of using dendritic cell-derived exosomes.

### 2. Description of Related Art

Dendritic cells (DC) are the master regulators of the mammalian adaptive immune response. In this capacity, DC sense T_{H} polarization cues in the surrounding environment and transmit this information to influence the character of downstream T-cell responses. However, DC are a relatively rare population of cells among circulating leukocytes. Patients typically undergo grueling mobilization regimens in order to generate sufficient numbers for therapeutic purposes. In addition, DC-based therapeutics are susceptible to disease-mediated immunosuppression and also run the risk of graft-versus-host disease. As such, compositions and methods that can provide the therapeutic benefits of DC without the risks associated therewith are needed.

### SUMMARY

As such, provided herein are therapeutic compositions comprising DC-derived extracellular vesicles (EV), which can be generated from significantly fewer DC than are needed to for DC-based therapeutics, thus lessening the requirement for mobilization regimens. Since DC-derived EV are subcellular entities without known intrinsic metabolic or signaling capabilities, they are spared from, e.g., tumor-mediated immunosuppression, unlike DC. Additionally, they are amenable for use as off-the-shelf therapies by virtue of the fact that they are subcellular entities with no known cytolytic capacity or the means to drive anti-EV immunity. As such, allogeneic EV can be delivered without the risk of GVHD or impactful host-versus-graft disease. Finally, EV display remarkable stability with regards to storage conditions. Unlike DC, which must be carefully cryopreserved and thawed in order to maintain function, EV remain intact and functional across a wide range of temperatures (e.g., from 25° C. to −80° C.).

Provided herein are methods generating extracellular vesicles (EV) from dendritic cells (DC), the methods comprising (i) maturing DC towards either T_{H}1 polarization or T_{H}2 polarization, and (ii) isolating EV secreted by the polarized DC. Maturing DC towards T_{H}1 polarization may comprise maturing the DC in the presence of IL-12 or loading the DC with lysate and mRNA preparations from the same cell type. Maturing DC towards T_{H}2 polarization may comprise maturing the DC in the presence of S. aureus enterotoxin B (SEB). The dendritic cells may be monocytic dendritic cells. The immature dendritic cells may be monocyte-derived dendritic cells. T_{H}1 polarized DC may be IL-12⁺, IL-13^{lo}, and CTLA-4^{lo}. T_{H}2 polarized DC may be IL-13⁺, CTLA-4⁺, and IL-12^{lo}. T_{H}1 polarized DC may be IL-12⁺, CADM1⁺, IL-13^{lo}, and CTLA-4^{lo}. T_{H}2 polarized DC may be IL-13⁺, CTLA-4⁺, CD172a⁺, and IL-12^{lo}. As used here, “lo” expression means a level of expression, as determined by qRT-PCR, that is at most 50% of the level as seen in oppositely polarized DC, e.g., the level of IL-13 in T_{H}1 polarized DC is at most 50% of the level of IL-13 in T_{H}2 polarized DC.

The methods may further comprise separating the secreted EV into CTLA-4⁺ and CTLA-4^{neg} populations. The CTLA-4^{neg} EV population is obtained by depletion of CTLA-4⁺ EV. The CTLA-4⁺ EV population is obtained by positive selection using bead-bound anti-CTLA-4 antibodies.

Provided herein are compositions comprising dendritic cell-derived extracellular vesicles (EV). The dendritic cell-derived EV may be obtained by the methods described herein. The EV may be CTLA-4^{neg} or CTLA-4⁺. The EV may be derived from either T_{H}1 polarized DC or T_{H}2 polarized DC. The EV may have a diameter of about 30-200 nm. The EV may be CD63⁺, CD81⁺, and HLA-DR⁺.

Provided herein are methods of separating CTLA-4⁺ and CTLA-4^{neg} EV from each other to recover both populations of EV in a state that is physically and functionally intact, the methods comprising isolating EV from mature DC cell culture supernatant using PEG precipitation and extracting the CTLA-4⁺ EV using bead-bound anti-CTLA-4 antibody, thereby CTLA-4⁺ and CTLA-4^{neg} EV from each other.

Provided herein are methods of inducing adaptive T_{H}1 polarization and activation of CD8⁺ T cells, the methods comprising contacting T cells with CTLA-4^{neg} DC-derived EV. The CTLA-4^{neg} DC-derived EV may be obtained according to a method described herein. The T cells may be present in bulk PBMC or they may be isolated naïve T cells. The T cells may be in a subject, in which case the methods comprise administering the EV to the subject.

Provided herein are methods of treating a disease in a patient in need thereof, the methods comprising administering CTLA-4^{neg} DC-derived EV to the patient. The disease may be a viral infection or a cancer. The DC-derived EV may be administered intratumorally, intravenously, peritumorally, subcutaneously, and intraperitoneally. The methods may further comprise administering an immune checkpoint inhibitor to the patient. The immune checkpoint inhibitor may be a CTLA-4 antagonist, such as, for example, ipilimumab, pembrolizumab or nivolumab.

Provided herein are methods of inhibiting adaptive T_{H}1 polarization and activation of T cells, the method comprising contacting the T cells with CTLA-4⁺ DC-derived EV. The T cells may be CD4⁺ T cells or CD8⁺ T cells. The EV may be derived from T_{H}2 polarized DC. The CTLA-4⁺ DC-derived EV may be obtained according to a method described herein. The T cells may be present in bulk PBMC or they may be isolated naïve T cells. The T cells may be in a subject, in which case the methods comprise administering the EV to the subject.

Provided herein are methods of treating an autoimmune condition in a patient, the methods comprising administering CTLA-4⁺ DC-derived EV to the patient. The autoimmune condition may be a T-cell mediated autoimmune condition. The autoimmune condition may be post-HSCT GVHD. The DC-derived EV may be administered intratumorally, intravenously, peritumorally, subcutaneously, and intraperitoneally.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B. FIG. 1A: Upregulated expression of CD8⁺CD161⁺ on T cells stimulated with T_{H}1 polarized vs. T_{H}2 polarized DC also showing downregulated levels of T-cell exhaustion markers PD-1 and Tim3. FIG. 1B: Addition of EVs derived from T_{H}2 polarized DC reduced the expression of IFN-γ and increases the expression of Tim3 on CD8⁺CD161⁺ T cells primed by T_{H}1 polarized DC.

FIG. 2. Total DC derived exosomes were separated into CTLA-4⁺ and CTLA-4^{neg} fractions and these were cultured with total non-adherent allogeneic PBMC as indicated for 7 days and analyzed by flow cytometry as indicated. PBMC culture with CTLA-4^{neg} exosomes elevated CD161 expression on the CD8⁺ cell population whereas CTLA-4⁺ exosomes tended to inhibit CD161 expression. CD8⁺CD161⁺ cells cultured with CTLA-4^{neg} exosomes substantially upregulated granzyme B and CD25 whereas CD8⁺CD161⁺ cells cultured with CTLA-4⁺ exosomes remained largely granzyme B low and CD25 negative.

FIGS. 3A-3B. Impact of DC CTLA-4⁺ EV secretion on downstream T-cell responses. FIG. 3A: Only T_{H}1 polarized DC secreting low or absent levels of CTLA-4⁺ EV can generate CD8⁺NK1.1⁺ T-cells. Adoptive transfer of 250,000 polarized DC to mice adjuvanted with the TLR-7 agonist imiquimod demonstrated that only DC secreting low levels of CTLA-4 could generate detectable levels of CD8⁺NK1.1⁺ highly cytolytic effector memory T-cells. A ten-fold increase in CD8⁺NK1.1⁺ T-cells was observed only among mice adoptively transferred with CTLA-4^{lo} T_{H}1 polarized DC. n=5-10 mice per group. FIG. 3B: CTLA-4 hi microvesicles suppress the generation of IFN-γ⁺ CD8+ T-cells in vitro. CTLA-4 hi microvesicles derived from T_{H}2 polarized DC culture supernatants were added to cocultures of T_{H}1 polarized DC and responder autologous T cells. Addition of these CTLA-4 hi microvesicles was sufficient to reduce the number of CD8⁺IFN-γ⁺ responders by 50%. ***p<0.001 by one-way ANOVA with Tukey's post-hoc.

FIGS. 4A-4C. CTLA-4^{neg} EV promote T_{H}1 T-cell polarization whereas CTLA-4⁺ EV inhibit T-cell T_{H}1 polarization. DCs used for exosome isolation were T_{H}2 polarized DCs (loaded with heterologous class I and class II peptides). All EVs derived from these DCs were added to buffy coat-derived non-adherent PBMC in culture. Either total EV or isolated CTLA-4^{neg} EV were used as indicated. FIG. 4A: Day 2 analysis of total CD4⁺ cells. FIG. 4B: Day 2 analysis of total CD8⁺ cells. FIG. 4C: Day 6 analysis of total CD8⁺ cells. Flow cytometry carried out on day 6 after the addition of IL-2 on day 5. Bulk PBMC were stimulated with PMA+Ionomycin+Brefeldin-A for 5 hours prior to flow staining. For day 6 staining, PBMC were kept overnight at 4 deg C. and stained the following day. Statistical analyses: One-Way ANOVA with Tukey's post-test. *, p≤0.05; **, p≤0.01; ***, p≤0.001; ****, p≤0.0001.

FIGS. 5A-5E. CTLA4^{neg} EVs upregulate cytotoxic CD8⁺CD161⁺ levels and functions. DC were polarized toward T_{H}1 by maturation in the presence of IL-12 or toward T_{H}2 by maturation in the presence of SEB. CTLA-4⁺ and CTLA-4^{neg} EV were isolated from both the T_{H}1 polarized and T_{H}2 polarized DC. All four EV populations were co-cultured separately with magnetically isolated CD45RA⁺ naïve allogeneic T cells for five days, after which T-cell populations were “restimulated” with another bolus of the same EV population. Flow cytometry analysis was performed on culture day 13. (FIG. 5A) Coculture of EVs isolated from the supernatant of T_{H}1 polarized DC with naïve T cells. The IFNγ and Granzyme B levels were measured in the CD8⁺CD161⁺ population. (FIG. 5B) Coculture of EVs isolated from the supernatant of T_{H}2 polarized DC with naïve T cells. The IFNγ and Granzyme B levels were measured in the CD8⁺CD161⁺ population. (FIG. 5C) IFN-γ and CD25 expression levels among the CD8+CD161+ T cells. Top panel: EV isolated from T_{H}1 polarized DC. Bottom panel: EV isolated from T_{H}2 polarized DC. (FIG. 5D) Granzyme B and CD25 expression levels among total CD8⁺ T cells. Top panel: EV isolated from T_{H}1 polarized DC. Bottom panel: EV isolated from T_{H}2 polarized DC. (FIG. 5E) IFN-γ and CD25 expression levels among total CD8⁺ T cells. Top panel: EV isolated from T_{H}1 polarized DC. Bottom panel: EV isolated from T_{H}2 polarized DC. No functional differences were noted among CTLA-4⁺ nor CTLA-4^{neg} EV populations whether isolated from T_{H}1 polarized DC or T_{H}2 polarized DC.

FIGS. 6A-6G. Maturation conditions modulate extravesicular CTLA-4 expression by human monocyte-derived DCs (moDCs). Human monocytes were isolated by positive CD14 immunomagnetic selection followed by culture with IL-4 and GM-CSF for 6 days. On day 6, immature DCs were matured and polarized as indicated. After 48 hours, mature DCs were characterized by: (FIG. 6A) qRT-PCR of IL-12a, IL-12b and IL-13 while EVs were characterized by (FIG. 6E) Western blot analysis; (FIG. 6B) miRNA Sequencing; (FIG. 6C) qRT-PCR of CTLA-4; (FIG. 6G) Cryo-Electron microscopy; (FIG. 6D) Mass spectrometry and (FIG. 6F) Flow cytometry. Statistical analysis by One-way ANOVA with Tukey's post-test. **, p≤0.01; ***, p≤0.001; ****, p≤0.0001.

FIGS. 7A-7L. EVs mirror parental DC phenotype to polarize T cell responses. (FIG. 7A) Allogeneic T cells were cultured with CFSE-labeled, Th-polarized and matured DC for 5 days. EV uptake (CFSE positivity) was assayed via flow cytometry. Allogeneic T cells were cultured with CFSE-labeled mature Th0-polarized DC for 3 days followed by flow cytometry to measure (FIG. 7B-7C) EV uptake by CD4 and CD8 T cells and (FIGS. 7D-7G) differential cytokine expression by CD4 and CD8 T cells based on EV uptake status. (FIG. 7H) Schematic detailing experimental design for data presented in panels FIGS. 71-7L in which allogeneic T cells were cultured with Cell Trace Far Red (CTFR)-labeled Th0-polarized DC and EV derived from CFSE-labeled Th1-polarized DC culture supernatant. On day 3, flow cytometry analysis was carried out to assay (FIG. 7I) EV uptake; (FIGS. 7J-L) T cell effector molecule expression. Statistical analysis by paired T-test or One-way ANOVA with Holm-Šídák's multiple comparisons test. *, p≤0.05; **, p≤0.01; ***, p≤0.001; ****, p≤0.0001.

FIGS. 8A-8F. CTLA-4+ EV suppress IFNγ expression by T cells. (FIG. 8A) Allogeneic T cells were cultured with CFSE-labeled, Th0-polarized DC in the presence or absence of anti-CTLA-4 (clone: BNI3) and EV uptake was assayed by flow cytometry analysis. (FIGS. 8B-8D) Allogeneic T cells were cultured with Total Th2 DC-derived EV or such EV which had been immunomagnetically depleted of the CTLA-4+ subset. T cell effector molecule expression was analyzed by flow cytometry on day 6. (FIGS. 8E-8F) CFSE-labeled DC were cultured with T cells in the presence of anti-CTLA-4 and assayed for uptake of dexosomes. Statistical analysis by One-way ANOVA with Dunn's post-test. *, p≤0.05; **, p≤0.01; *** p≤0.001; ****, p≤0.0001.

FIGS. 9A-9D. Loss of DC CTLA-4 drives T cell activation and lethality in mice. (FIG. 9A) C57BL/6 mice were implanted with B16-Flt3L tumors and sacrificed for splenocyte isolation on day 12. CTLA-4 expression by monocyte and DC subsets were assayed by flow cytometry analysis. (FIG. 9B-9C) Splenic T cells from wildtype (WT) C57BL/6 or CTLA-4^{fl/fl} CD11c^{cre} mice (cKO) were characterized by flow cytometry for activation state and CTLA-4 expression. (FIG. 9D) H&E staining was performed on lymphoid and non-lymphoid Tissues of WT or cKO mice. White arrows indicate regions of leukocyte infiltration.

FIG. 10. Loss of EV CTLA4 promotes anti-tumor T cell responses. In the survival curve, the lines represent OVA siCTLA4, OVA siNT, IL-12 siNT, and PBS, from top to bottom at day 30.

### DETAILED DESCRIPTION

Dendritic cells (DC) are the master regulators of the mammalian adaptive immune response. In this capacity, DC sense T_{H} polarization cues in the surrounding environment and transmit this information to influence the character of downstream T-cell responses. DC transmit this information to T cells through the secretion of exosomes or extracellular vesicles (EV). T_{H}2 polarized DC preferentially secrete EV bearing the immunoregulatory checkpoint molecule CTLA-4, whereas T_{H}1 polarized DC preferentially secrete EV that are devoid of this protein. Provided herein are methods to generate EV from cultures of polarized DC as well as additional procedures to separate CTLA-4⁺ and CTLA-4^{neg} EV from each other and recover both populations of EV in a state that is physically and functionally intact. In vitro culture of T cells with CTLA-4⁺ EV inhibits adaptive T_{H}1 polarization and activation of CD8⁺ T cells, whereas culture with CTLA-4^{neg} EV induces adaptive T_{H}1 polarization and activation of CD8⁺ T cells. CTLA-4^{neg} EV also induce the generation of a critical CD8⁺ T-cell memory population identified by the expression of CD161. These effects are seen with both bulk PBMC and magnetically isolated naïve T cells. Large scale production and direct administration of these EV is expected to promote the development of CD8⁺ T cells for the treatment of cancer or for the inhibition of activated T cells to treat autoimmune conditions including post-HSCT GVHD. As such, the use of CTLA-4^{neg} EV for the treatment of cancer, and the use of CTLA-4⁺ EV for the treatment of GVHD and other T-cell mediated autoimmune conditions, are provided herein.

### I. Definitions

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

The term “essentially” is to be understood that methods or compositions include only the specified steps or materials and those that do not materially affect the basic characteristics of those methods and compositions.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

The term “effective,” as that term is used in the specification and/or claims, means adequate to accomplish a desired, expected, or intended result. “Effective amount,” “therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

### II. Dendritic Cell Extracellular Vesicles

Methods for isolating, culturing, and priming dendritic cells, from which DC-derived EVs can be isolated, are well known in the art. For example, U.S. Pat. No. 8,728,806, which is incorporated herein by reference in its entirety, provides detailed methods for providing antigen primed dendritic cells. In certain aspects, dendritic cells for use according to the embodiments are isolated from a subject that is to be treated by a method of the embodiments. In other aspects, dendritic cells may be from a different subject, such as an HLA-matched donor. In certain aspects, the dendritic cells are from a bank of dendritic cells having a defined HLA typing.

Methods for isolating cell populations enriched for dendritic cell precursors and immature dendritic cells from various sources, including blood and bone marrow, are known in the art. For example, dendritic cell precursors and immature dendritic cells can be isolated by collecting heparinized blood, by apheresis or leukapheresis, by preparation of buffy coats, rosetting, centrifugation, density gradient centrifugation (e.g., using Ficoll (such as FICOLL-PAQUE®), PERCOLL® (colloidal silica particles (15-30 mm diameter) coated with non-dialyzable polyvinylpyrrolidone (PVP)), sucrose, and the like), differential lysis of cells, filtration, and the like. In certain embodiments, a leukocyte population can be prepared, such as, for example, by collecting blood from a subject, defibrinating to remove the platelets, and lysing the red blood cells. In other words, a leukocyte population can be prepared, such as, for example, by defibrinating a blood sample to remove the platelets and lysing the red blood cells. Dendritic cell precursors and immature dendritic cells can optionally be enriched for monocytic dendritic cell precursors by, for example, centrifugation through a PERCOLL® gradient. In other aspects, dendritic cell precursors can be selected using CD14 selection of G-CSF mobilized peripheral blood. In still other aspects, monocytic dendritic cell precursors can be selected by size through differential centrifugation procedures sometimes referred to as elutriation.

Dendritic cell precursors and immature dendritic cells optionally can be prepared in a closed, aseptic system. As used herein, the terms “closed, aseptic system” or “closed system” refer to a system in which exposure to non-sterilize, ambient, or circulating air or other non-sterile conditions is minimized or eliminated. Closed systems for isolating dendritic cell precursors and immature dendritic cells generally exclude density gradient centrifugation in open top tubes, open air transfer of cells, culture of cells in tissue culture plates or unsealed flasks, and the like. In a typical embodiment, the closed system allows aseptic transfer of the dendritic cell precursors and immature dendritic cells from an initial collection vessel to a sealable tissue culture vessel without exposure to non-sterile air.

In certain embodiments, monocytic dendritic cell precursors are isolated by adherence to a monocyte-binding substrate. For example, a population of leukocytes (e.g., isolated by leukapheresis) can be contacted with a monocytic dendritic cell precursor adhering substrate. When the population of leukocytes is contacted with the substrate, the monocytic dendritic cell precursors in the leukocyte population preferentially adhere to the substrate. Other leukocytes (including other potential dendritic cell precursors) exhibit reduced binding affinity to the substrate, thereby allowing the monocytic dendritic cell precursors to be preferentially enriched on the surface of the substrate.

Suitable substrates include, for example, those having a large surface area to volume ratio. Such substrates can be, for example, a particulate or fibrous substrate. Suitable particulate substrates include, for example, glass particles, plastic particles, glass-coated plastic particles, glass-coated polystyrene particles, and other beads suitable for protein absorption. Suitable fibrous substrates include microcapillary tubes and microvillous membrane. The particulate or fibrous substrate usually allows the adhered monocytic dendritic cell precursors to be eluted without substantially reducing the viability of the adhered cells. A particulate or fibrous substrate can be substantially non-porous to facilitate elution of monocytic dendritic cell precursors or dendritic cells from the substrate. A “substantially non-porous” substrate is a substrate in which at least a majority of pores present in the substrate are smaller than the cells to minimize entrapping cells in the substrate.

Adherence of the monocytic dendritic cell precursors to the substrate can optionally be enhanced by addition of binding media. Suitable binding media include monocytic dendritic cell precursor culture media (e.g., AIM-V®, RPMI 1640, DMEM, X-VIVO 15®, and the like) supplemented, individually or in any combination, with for example, cytokines (e.g., Granulocyte/Macrophage Colony Stimulating Factor (GM-CSF), Interleukin 4 (IL-4)), blood plasma, serum (e.g., human serum, such as autologous or allogenic sera), purified proteins, such as serum albumin, divalent cations (e.g., calcium and/or magnesium ions) and other molecules that aid in the specific adherence of monocytic dendritic cell precursors to the substrate, or that prevent adherence of non-monocytic dendritic cell precursors to the substrate. In certain embodiments, the blood plasma or serum can be heated-inactivated. The heat-inactivated plasma can be autologous or heterologous to the leukocytes.

Following adherence of monocytic dendritic cell precursors to the substrate, the non-adhering leukocytes are separated from the monocytic dendritic cell precursor/substrate complexes. Any suitable means can be used to separate the non-adhering cells from the complexes. For example, the mixture of the non-adhering leukocytes and the complexes can be allowed to settle, and the non-adhering leukocytes and media decanted or drained. Alternatively, the mixture can be centrifuged, and the supernatant containing the non-adhering leukocytes decanted or drained from the pelleted complexes.

Isolated dendritic cell precursors can be cultured ex vivo for differentiation, maturation, and/or expansion. As used herein, isolated immature dendritic cells, dendritic cell precursors, T cells, and other cells, refers to cells that, by human hand, exists apart from their native environment, and are therefore not a product of nature. Isolated cells can exist in purified form, in semi-purified form, or in a non-native environment. Briefly, ex vivo differentiation typically involves culturing dendritic cell precursors, or populations of cells having dendritic cell precursors, in the presence of one or more differentiation agents. Suitable differentiating agents can be, for example, cellular growth factors (e.g., cytokines such as (GM-CSF), Interleukin 4 (IL-4), and/or combinations thereof). In certain embodiments, the monocytic dendritic cells precursors are differentiated to form monocyte-derived immature dendritic cells.

The dendritic cell precursors can be cultured and differentiated in suitable culture conditions. Suitable tissue culture media include AIM-V®, RPMI 1640, DMEM, X-VIVO 15®, and the like. The tissue culture media can be supplemented with serum, amino acids, vitamins, cytokines, such as GM-CSF and/or IL-4, divalent cations, and the like, to promote differentiation of the cells. In certain embodiments, the dendritic cell precursors can be cultured in the serum-free media. Such culture conditions can optionally exclude any animal-derived products. A typical cytokine combination in a typical dendritic cell culture medium is about 500 units/ml each of GM-CSF (50 ng/ml) and IL-4 (10 ng/ml). Dendritic cell precursors, when differentiated to form immature dendritic cells, are phenotypically similar to skin Langerhans cells. Immature dendritic cells typically are CD14^{neg} and CD11c⁺, express absent levels of CD80 and CD83 and are able to capture soluble antigens via specialized endocytosis. Immature DC express high levels of CD86.

The immature dendritic cells are matured to form mature dendritic cells. Mature DC lose the ability to take up antigen and display up-regulated expression of costimulatory cell surface molecules and various cytokines. Specifically, mature DC express higher levels of MHC class I and II antigens than immature dendritic cells, and mature dendritic cells are generally identified as being CD80⁺, CD83⁺, CD86⁺, and CD14^{neg}. Greater MHC expression leads to an increase in antigen density on the DC surface, while up regulation of costimulatory molecules CD80 and CD86 strengthens the T-cell activation signal through the counterparts of the costimulatory molecules, such as CD28 on the T cells.

For example, mononuclear cells (MNCs) separated from mobilized (e.g., granulocyte colony stimulating factor (G-CSF) mobilized) peripheral blood progenitor cells by centrifugation on a Histopaque-1077 (Sigma, St. Louis, MO) gradient for 20 min at 450×g. Following separation, CD14⁺ MNCs can be isolated on a magnetic separation column with CD14 Microbeads according to the manufacturer's instructions (Miltenyi Biotec). CD14⁺ cells may be cultured for 6 days, in a humidified chamber at 37° C. and 5% atmospheric CO₂, at a concentration of 2×10⁶ cells/ml in AIM-V medium (Invitrogen, Carlsbad, CA) supplemented with 10% Human AB Serum (Atlanta Biologicals, Lawrenceville, GA), 50 μg/ml streptomycin sulfate (Invitrogen), 10 μg/ml gentamicin sulfate, 2 mM L-glutamine (Invitrogen), 50 ng/ml GM-CSF (Amgen, Thousand Oaks, CA), and 10 ng/ml IL-4 (R&D Systems, Minneapolis, MN). The culture medium may be removed and replenished with an equal volume of fresh medium on day 3. After 6 days of culture in GM-CSF and IL-4, immature DC are harvested using an enzyme-free cell dissociation buffer. Immature DC display the phenotype of CD11c⁺, CD80^{neg}, CD83^{neg}, CD86⁺, CD209⁺, and HLA-DR⁺ as determined by flow cytometry.

Immature DC may be loaded with individual recombinant proteins and/or transduced with any vector expressing the same. For example, immature DC can be loaded with either tumor lysate, tumor mRNA, or both. If loading with lysate, an exemplary protocol is as follows: immature DC are cultured for three hours at 37° C. at a concentration of 5×10⁶ cells/ml in a four-fold dilution of cell lysate. Following lysate loading, cells are pelleted, washed once to remove residual lysate, and matured at a density of 1-2×10⁶ cells/ml for 36-48 h in AIM-V medium (Invitrogen, Carlsbad, CA) supplemented with 10% Human AB Serum (Atlanta Biologicals, Lawrenceville, GA), 50 μg/ml streptomycin sulfate (Invitrogen), 10 μg/ml gentamicin sulfate, 2 mM L-glutamine (Invitrogen), 50 ng/ml GM-CSF (Amgen, Thousand Oaks, CA), 10 ng/ml IL-4 (R&D Systems, Minneapolis, MN), and with the addition of ITIP [10 ng/ml IL-1β (R&D Systems), 10 ng/ml TNF-α (R&D Systems), 15 ng/ml IL-6 (R&D Systems), and 1 μg/ml PGE2 (Sigma)] (see Decker et al., Vaccine 24:3203-3216, 2006). In order to stimulate T_{H}2 polarization of the DC, the medium is, for example, further supplemented with 10 ng/ml SEB (see Halpert et al., Stem Cells and Development 25:774-787, 2016). T_{H}2 polarized DC display the phenotype of expressing low levels of IL-12 and high levels of IL-13, GATA3, CTLA-4, and SIRP-alpha (CD172a), as measured by qPCR. In order to stimulate T_{H}1 polarization of the DC, the medium is, for example, further supplemented with 1-2 ng/ml IL-12. T_{H}1 polarized DC display the phenotype of expressing low levels of IL-13 and CTLA-4 and high levels of IL-12 and CADM1, as measured by qPCR.

For loading with mRNA, an exemplary electroporation protocol is as follows: immature DC are suspended at a concentration of 4×10⁷ cells/ml in Viaspan (Barr Laboratories, Pomona, NY), mixed with tumor mRNA to a concentration of 1 μg mRNA/10⁶ cells, and incubated for 10 min on ice in an electroporation cuvette with an 0.4 cm gap (Biorad, Hercules, CA). Cells are then electroporated at 300V, 150 μF, and Ω=∞ using a GenePulser Xcell (Biorad). Following electroporation, cells are immediately introduced into culture and treated identically to lysate-loaded DCs. DC viability post-electroporation is typically greater than 75%.

Double loaded DC may receive lysate and mRNA preparations from the same cell type, thus inducing T_{H}1 polarization. When loaded with both mRNA and lysate, cells may receive either lysate first and mRNA second or mRNA first and lysate second. Preferably, when loaded with both mRNA and lysate, cells receive mRNA first and lysate second in order to avoid exposure of the mRNA to any RNAses that may be present in the lysate.

Following loading and maturation, DC display the phenotype of CD11c⁺, CD80⁺, CD83⁺, CD86⁺⁺, CD209⁺, and HLA-DR⁺⁺. Note that markers involved with co-stimulation or antigen presentation are upregulated upon maturation (CD80, CD83, CD86, HLA-DR), whereas markers involved with adhesion are downregulated (CD11c, CD209).

Mature dendritic cells of the present invention can be prepared (i.e., matured) by contacting the immature dendritic cells with effective amounts or concentrations of a nucleic acid composition and a homologous cell antigen composition. Effective amounts of nucleic acid composition typically range from at most, at least, or about 0.01, 0.1, 1, 5, 10, to 10, 15, 20, 50, 100 ng or mg of nucleic acid per culture dish or per cell, including all values and ranges there between. Effective amounts of cell lysate antigenic composition typically range from at most, at least, or about 0.01, 0.1, 1, 5, 10, to 10, 15, 20, 50, 100 ng or mg of protein per culture dish or per cell. In certain aspects 0.001 ng of tumor antigen/cell to 1 μg of cell lysate antigen/million cells) can be used. The cell lysate antigenic composition can optionally be heat inactivated, irradiated, or treated (e.g., exposed to protease) prior to contact with DC.

The immature DC are typically contacted with effective amounts of a nucleic acid composition and a cell lysate antigenic composition for at most, at least, or about 1, 2, 3, 4, 5, 6, 7, 8, 9, 10, 11, 12, to 10, 11, 12, 13, 14, 15, 16, 17, 18, 19, 20, 21, 22, 23, or 24 minutes, hours, or days. The immature DC can be cultured and matured in suitable maturation culture conditions. Suitable tissue culture media include AIM-V®, RPMI 1640, DMEM, X-VIVO 15®, and the like. The tissue culture media can be supplemented with amino acids, vitamins, cytokines, such as GM-CSF and/or IL-4, divalent cations, and the like, to promote maturation of the cells.

Maturation of dendritic cells can be monitored by methods known in the art. Cell surface markers can be detected in assays familiar to the art, such as flow cytometry, immunohistochemistry, and the like. The cells can also be monitored for cytokine production (e.g., by ELISA, FACS, or other immune assay). Dendritic cell precursors, immature dendritic cells, and mature dendritic cells, either primed or unprimed, with antigens can be cryopreserved for use at a later date. Methods for cryopreservation are well-known in the art. For example, U.S. Pat. No. 5,788,963, which is incorporated herein by reference in its entirety.

In order to isolate EV from mature DC, cell culture supernatants are subjected to sequential centrifugation at 400×g for 10 minutes and then 2000×g for 30 minutes at room temperature in order to remove pelleted cells and cellular debris from the preparation. The EV present in the resulting supernatant are precipitated using PEG precipitation. For example, the resulting supernatant is incubated with cold Total Exosome Isolation Reagent (Invitrogen cat no.: 4478359) for 16-18 hours. Subsequently, this mixture is spun down at 10,000×g for 1 hour at 4° C. The resulting pellet is resuspended in PBS and incubated with magnetic bead-bound anti-CTLA-4 for 16-18 hours at 4° C. to facilitate CTLA-4⁺ EV isolation/depletion by the application of a strong magnetic field. Recovery of intact CTLA-4⁺ EVs is performed via high-salt buffer elution for 2 hours at room temperature. The isolated EV may have a diameter of 30-200 nm, as measured by cryo-EM. The EV, regardless of CTLA-4 status, display the phenotype of CD63⁺, CD81⁺, and HLA-DR⁺.

### III. Compositions and Methods for Treatment

Some aspects of the embodiments concern administration of CTLA-4⁺ or CTLA-4^{neg} EV isolated from mature dendritic cells. In particular, for the development of CD8⁺ T cells for the treatment of cancer, CTLA-4^{neg} EV are to be administered. On the other hand, for the inhibition of activated T cells to treat T-cell mediated autoimmune conditions, such as, for example, post-HSCT GVHD, CTLA-4⁺ EV are to be administered.

In the context of treating T-cell mediated autoimmune conditions, such conditions that may be treated according to the methods described herein include, but are not limited to, post-hematopoietic stem cell transplantation (post-HSCT) graft-versus-host disease (GVHD), psoriasis, lichen planus, vitiligo, autoimmune type 1 diabetes mellitus (T1DM), rheumatoid arthritis, multiple sclerosis, celiac disease, and MHC-associated inflammatory eye disease (e.g., idiopathic uveitis, birdshot retinochoroidopathy (BSR), and sympathetic ophthalmia). Further, examples of autoimmune diseases include, but are not limited to, systemic lupus erythematosus (SLE), Sjögren's syndrome, Hashimoto's thyroiditis, Graves' disease, idiopathic thrombocytopenic purpura, acute idiopathic thrombocytopenia purpura, chronic idiopathic thrombocytopenic purpura, dermatomyositis, Sydenham's chorea, myasthenia gravis, lupus nephritis, rheumatic fever, polyglandular syndromes, bullous pemphigoid, Henoch-Schonlein purpura, post-streptococcalnephritis, erythema nodosurn, Takayasu's arteritis, Addison's disease, sarcoidosis, ulcerative colitis, erythema multiforme, IgA nephropathy, polyarteritis nodosa, ankylosing spondylitis, Goodpasture's syndrome, thromboangitisubiterans, primary biliary cirrhosis, thyrotoxicosis, scleroderma, chronic active hepatitis, polymyositis/dermatomyositis, polychondritis, pamphigus vulgaris, Wegener's granulomatosis, membranous nephropathy, amyotrophic lateral sclerosis, tabes dorsalis, giant cell arteritis/polymyalgia, perniciousanemia, rapidly progressive glomerulonephritis, psoriasis, and fibrosing alveolitis.

In the context of treating cancer, cancers that may be treated according to the methods described herein include, but are not limited to, cancers of the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In some aspects, the cancer may be a neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In further aspects, the cancer is a brain cancer (e.g., a glioma), a prostate cancer, a breast cancer (e.g., a triple negative breast cancer), a pancreatic cancer (e.g., a pancreatic ductal adenocarcinoma), acute myeloid leukemia (AML), melanoma, renal cell cancer or chronic lymphocytic leukemia.

In certain aspects, the EV for use in the methods are suitably contained in a pharmaceutically acceptable carrier. The carrier is non-toxic, biocompatible and is selected so as not to detrimentally affect the biological activity of the EV. The EV may be formulated into preparations for local delivery (i.e. to a specific location of the body, such as skeletal muscle or other tissue) or systemic delivery. Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose, any biocompatible oil may be employed including synthetic mono- or diglycerides. In addition, fatty acids such as oleic acid find use in the preparation of injectables.

Solutions of pharmaceutical compositions can be prepared in water suitably mixed with a surfactant, such as hydroxypropylcellulose. The compositions of the disclosure may comprise glycerol, liquid polyethylene glycols, and mixtures thereof. Under ordinary conditions of storage and use, these preparations contain a preservative to prevent the growth of microorganisms.

In certain aspects, the pharmaceutical compositions are advantageously administered in the form of injectable compositions either as liquid solutions or suspensions. A typical composition for such purpose comprises a pharmaceutically acceptable carrier. For instance, the composition may contain less, than, equal to, or more than 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-fungal agents, anti-oxidants, chelating agents and inert gases. The pH and exact concentration of the various components the pharmaceutical composition are adjusted according to well-known parameters.

In further aspects, the pharmaceutical compositions comprising EV may include classic pharmaceutical preparations. In some embodiments, the compositions can be administered to a subject by any method known to those of ordinary skill in the art. Examples include intravenously, intradermally, intraarterially, intraperitoneally, intralesionally, intratumorally, peritumorally intramuscularly, subcutaneously, intravesicularlly, mucosally, by injection, by infusion, by continuous infusion, via a catheter, or by other method or any combination of the forgoing as would be known to one of ordinary skill in the art. Administration of pharmaceutical compositions comprising EV according to certain aspects may be via any common route so long as the target tissue is available via that route. Administration may be by orthotopic, intradermal, subcutaneous, intramuscular, intratumoral, peritumoral, intraperitoneal or intravenous injection. Such compositions would normally be administered as pharmaceutically acceptable compositions that include physiologically acceptable carriers, buffers or other excipients. In certain aspects of the embodiments, EV are administered to a lymphoid tissue site proximal to the diseased cell population in the subject. The lymphoid tissue site may be a lymphoid tissue that drains tissue surrounding the diseased cell population. For example, EV are, in some aspects, administered to a lymph node that that drains tissue surrounding the diseased cell population. In some aspects, EV are administered to a tissue that drains into the lymphoid tissue site proximal to the diseased cell population in the subject.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment {e.g., alleviation of symptoms versus cure) and the potency, stability and toxicity of the particular therapeutic substance. In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% (w/v) of an active agent, such as an isolated EV. In other embodiments, the EV may comprise between about 2% (w/v) to about 75% (w/v) of the unit, or between about 25% (w/v) to about 60% (w/v), for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 microgram/kg/body weight to about 100 mg/kg/body weight, about 5 microgram/kg/body weight to about 500 milligram/kg/body weight, etc., can be administered.

Methods may involve administering a composition containing (or a composition comprising) about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 445, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 nanograms (ng), micrograms (meg), milligrams (mg), or grams of exosomes, or any range derivable therein. The above numerical values may also be the dosage that is administered to the patient based on the patient's weight, expressed as ng/kg, mg/kg, or g/kg, and any range derivable from those values.

Alternatively, the composition may have a concentration of exosomes that are 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000 ng/ml, μg/ml, mg/ml, or g/ml, or any range derivable therein.

The composition may be administered to (or taken by) the patient 1, 2, 3, 4, 5, 6, 7, 8, 9, 10, 11, 12, 13, 14, 15, 16, 17, 18, 19, 20 or more times, or any range derivable therein, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months, or any range derivable therein. It is specifically contemplated that the composition may be administered once daily, twice daily, three times daily, four times daily, five times daily, or six times daily (or any range derivable therein) and/or as needed to the patient. Alternatively, the composition may be administered every 2, 4, 6, 8, 12 or 24 hours (or any range derivable therein) to or by the patient. In some embodiments, the patient is administered the composition for a certain period of time or with a certain number of doses after experiencing symptoms of a demyelinating disorder.

### IV. Combination Therapies

In order to increase the effectiveness of dendritic cell-derived EV therapies, it may be desirable to combine these compositions with other agents effective in the treatment of the disease of interest.

As a non-limiting example, the treatment of cancer may be implemented with a primed dendritic cell composition of the present embodiments along with other anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the anti-cancer peptide or nanoparticle complex and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the dendritic cell composition and the other includes the second agent(s).

Treatment with a dendritic cell composition may precede or follow the other agent treatment by intervals ranging from minutes to weeks. In embodiments where the other agent and dendritic cell composition are applied separately to the subject, one would generally ensure that a significant period of time did not expire between the time of each delivery, such that the agent and the dendritic cell composition would still be able to exert an advantageously combined effect on the cell. In such instances, it is contemplated that one may contact the cell with both modalities within about 12-24 hours of each other and, more preferably, within about 6-12 hours of each other. In some situations, it may be desirable to extend the time period for treatment significantly where several days (e.g., 2, 3, 4, 5, 6 or 7 days) to several weeks (e.g., 1, 2, 3, 4, 5, 6, 7 or 8 weeks) lapse between the respective administrations.

Various combinations may be employed, where dendritic cell-derived MV therapy is “A” and the secondary agent, such as radiotherapy, chemotherapy or anti-inflammatory agent, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

In certain embodiments, administration of dendritic cell therapy of the present embodiments to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

### A. Chemotherapy

Cancer therapies also include a variety of combination therapies. In some aspects a dendritic cell-derived MV composition of the embodiments is administered (or formulated) in conjunction with a chemotherapeutic agent. For example, in some aspects the chemotherapeutic agent is a protein kinase inhibitor such as a EGFR, VEGFR, AKT, Erb1, Erb2, ErbB, Syk, Bcr-Abl, JAK, Src, GSK-3, PI3K, Ras, Raf, MAPK, MAPKK, mTOR, c-Kit, eph receptor or BRAF inhibitors. Nonlimiting examples of protein kinase inhibitors include Afatinib, Axitinib, Bevacizumab, Bosutinib, Cetuximab, Crizotinib, Dasatinib, Erlotinib, Fostamatinib, Gefitinib, Imatinib, Lapatinib, Lenvatinib, Mubritinib, Nilotinib, Panitumumab, Pazopanib, Pegaptanib, Ranibizumab, Ruxolitinib, Saracatinib, Sorafenib, Sunitinib, Trastuzumab, Vandetanib, AP23451, Vemurafenib, MK-2206, GSK690693, A-443654, VQD-002, Miltefosine, Perifosine, CAL101, PX-866, LY294002, rapamycin, temsirolimus, everolimus, ridaforolimus, Alvocidib, Genistein, Selumetinib, AZD-6244, Vatalanib, P1446A-05, AG-024322, ZD1839, P276-00, GW572016 or a mixture thereof.

Yet further combination chemotherapies include, for example, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaI1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; carboplatin, procarbazine, plicomycin, gemcitabien, navelbine, farnesyl-protein tansferase inhibitors, transplatinum, and pharmaceutically acceptable salts, acids or derivatives of any of the above. In certain embodiments, the compositions provided herein may be used in combination with gefitinib. In other embodiments, the present embodiments may be practiced in combination with Gleevac (e.g., from about 400 to about 800 mg/day of Gleevac may be administered to a patient). In certain embodiments, one or more chemotherapeutic may be used in combination with the compositions provided herein.

### B. Immunotherapy

The skilled artisan will understand that immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T-cells and NK cells.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune-stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds; cytokine therapy, e.g., interferons α, β, and γ, IL-1, GM-CSF, and TNF; gene therapy, e.g., TNF, IL-1, IL-2, and p53; and monoclonal antibodies, e.g., anti-CD20, anti-ganglioside GM2, and anti-p185. It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Immune checkpoint proteins that may be targeted by immune checkpoint blockade include adenosine A2A receptor (A2AR), B7-H3 (also known as CD276), B and T lymphocyte attenuator (BTLA), CCL5, CD27, CD38, CD8A, CMKLR1, cytotoxic T-lymphocyte-associated protein 4 (CTLA-4, also known as CD152), CXCL9, CXCR5, glucocorticoid-induced tumour necrosis factor receptor-related protein (GITR), HLA-DRB1, ICOS (also known as CD278), HLA-DQA1, HLA-E, indoleamine 2,3-dioxygenase 1 (IDO1), killer-cell immunoglobulin (KIR), lymphocyte activation gene-3 (LAG-3, also known as CD223), Mer tyrosine kinase (MerTK), NKG7, OX40 (also known as CD134), programmed death 1 (PD-1), programmed death-ligand 1 (PD-L1, also known as CD274), PDCD1LG2, PSMB10, STAT1, T-cell immunoreceptor with Ig and ITIM domains (TIGIT), T-cell immunoglobulin domain and mucin domain 3 (TIM-3), V-domain Ig suppressor of T-cell activation (VISTA, also known as C10orf54), and 4-1BB (CD137). In particular, the immune checkpoint inhibitors target the PD-1 axis and/or CTLA-4.

The immune checkpoint inhibitors may be drugs, such as small molecules, recombinant forms of ligand or receptors, or antibodies, such as human antibodies (e.g., International Patent Publication WO2015/016718; Pardoll, Nat Rev Cancer, 12 (4): 252-264, 2012; both incorporated herein by reference). Known inhibitors of the immune checkpoint proteins or analogs thereof may be used, in particular, chimeric, humanized, or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, a PD-1 binding antagonist is a molecule that inhibits the binding of PD-1 to its ligand binding partners. In a specific aspect, the PD-1 ligand binding partners are PD-L1 and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, a PD-L2 binding antagonist is a molecule that inhibits the binding of PD-L2 to its binding partners. In a specific aspect, a PD-L2 binding partner is PD-1. The antagonist may be an antibody, an antigen-binding fragment thereof, an immunoadhesin, a fusion protein, or an oligopeptide. Exemplary antibodies are described in U.S. Pat. Nos. 8,735,553, 8,354,509, and 8,008,449, all of which are incorporated herein by reference. Other PD-1 axis antagonists for use in the methods provided herein are known in the art, such as described in U.S. Patent Application Publication Nos. 2014/0294898, 2014/022021, and 2011/0008369, all of which are incorporated herein by reference.

In some embodiments, a PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342.

Another immune checkpoint protein that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T-cells and acts as an “off” switch when bound to CD80 or CD86 on the surface of antigen-presenting cells. CTLA-4 is similar to the T-cell co-stimulatory protein, CD28, and both molecules bind to CD80 and CD86, also called B7-1 and B7-2 respectively, on antigen-presenting cells. CTLA-4 transmits an inhibitory signal to T-cells, whereas CD28 transmits a stimulatory signal. Intracellular CTLA-4 is also found in regulatory T-cells and may be important to their function. T-cell activation through the T-cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide. Anti-human-CTLA-4 antibodies (or VH and/or VL domains derived therefrom) suitable for use in the present methods can be generated using methods well known in the art. Alternatively, art-recognized anti-CTLA-4 antibodies can be used. For example, the anti-CTLA-4 antibodies disclosed in U.S. Pat. No. 8,119,129; PCT Publn. Nos. WO 01/14424, WO 98/42752, WO 00/37504 (CP675,206, also known as tremelimumab; formerly ticilimumab); U.S. Pat. No. 6,207,156; Hurwitz et al. (1998) Proc Natl Acad Sci USA, 95 (17): 10067-10071; Camacho et al. (2004) J Clin Oncology, 22 (145): Abstract No. 2505 (antibody CP-675206); and Mokyr et al. (1998) Cancer Res, 58:5301-5304 can be used in the methods disclosed herein. The teachings of each of the aforementioned publications are hereby incorporated by reference. Antibodies that compete with any of these art-recognized antibodies for binding to CTLA-4 can also be used. For example, a humanized CTLA-4 antibody is described in International Patent Application No. WO2001/014424, WO2000/037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen-binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2, and CDR3 domains of the VL region of ipilimumab. In another embodiment, the antibody competes for binding with and/or binds to the same epitope on CTLA-4 as the above-mentioned antibodies. In another embodiment, the antibody has an at least about 90% variable region amino acid sequence identity with the above-mentioned antibodies (e.g., at least about 90%, 95%, or 99% variable region identity with ipilimumab). Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

Another immune checkpoint protein that can be targeted in the methods provided herein is lymphocyte-activation gene 3 (LAG-3), also known as CD223. The complete protein sequence of human LAG-3 has the Genbank accession number NP-002277. LAG-3 is found on the surface of activated T-cells, natural killer cells, B cells, and plasmacytoid dendritic cells. LAG-3 acts as an “off” switch when bound to MHC class II on the surface of antigen-presenting cells. Inhibition of LAG-3 both activates effector T-cells and inhibitor regulatory T-cells. In some embodiments, the immune checkpoint inhibitor is an anti-LAG-3 antibody (e.g., a human antibody, a humanized antibody, or a chimeric antibody), an antigen-binding fragment thereof, an immunoadhesin, a fusion protein, or oligopeptide. Anti-human-LAG-3 antibodies (or VH and/or VL domains derived therefrom) suitable for use in the present methods can be generated using methods well known in the art. Alternatively, art-recognized anti-LAG-3 antibodies can be used. An exemplary anti-LAG-3 antibody is relatlimab (also known as BMS-986016) or antigen binding fragments and variants thereof (see, e.g., WO 2015/116539). Other exemplary anti-LAG-3 antibodies include TSR-033 (see, e.g., WO 2018/201096), MK-4280, and REGN3767. MGD013 is an anti-LAG-3/PD-1 bispecific antibody described in WO 2017/019846. FS118 is an anti-LAG-3/PD-L1 bispecific antibody described in WO 2017/220569.

Another immune checkpoint protein that can be targeted in the methods provided herein is V-domain Ig suppressor of T-cell activation (VISTA), also known as C10orf54. The complete protein sequence of human VISTA has the Genbank accession number NP_071436. VISTA is found on white blood cells and inhibits T-cell effector function. In some embodiments, the immune checkpoint inhibitor is an anti-VISTA3 antibody (e.g., a human antibody, a humanized antibody, or a chimeric antibody), an antigen-binding fragment thereof, an immunoadhesin, a fusion protein, or oligopeptide. Anti-human-VISTA antibodies (or VH and/or VL domains derived therefrom) suitable for use in the present methods can be generated using methods well known in the art. Alternatively, art-recognized anti-VISTA antibodies can be used. An exemplary anti-VISTA antibody is JNJ-61610588 (also known as onvatilimab) (see, e.g., WO 2015/097536, WO 2016/207717, WO 2017/137830, WO 2017/175058). VISTA can also be inhibited with the small molecule CA-170, which selectively targets both PD-L1 and VISTA (see, e.g., WO 2015/033299, WO 2015/033301).

Another immune checkpoint protein that can be targeted in the methods provided herein is indoleamine 2,3-dioxygenase (IDO). The complete protein sequence of human IDO has Genbank accession number NP_002155. In some embodiments, the immune checkpoint inhibitor is a small molecule IDO inhibitor. Exemplary small molecules include BMS-986205, epacadostat (INCB24360), and navoximod (GDC-0919).

Another immune checkpoint protein that can be targeted in the methods provided herein is CD38. The complete protein sequence of human CD38 has Genbank accession number NP_001766. In some embodiments, the immune checkpoint inhibitor is an anti-CD38 antibody (e.g., a human antibody, a humanized antibody, or a chimeric antibody), an antigen-binding fragment thereof, an immunoadhesin, a fusion protein, or oligopeptide. Anti-human-CD38 antibodies (or VH and/or VL domains derived therefrom) suitable for use in the present methods can be generated using methods well known in the art. Alternatively, art-recognized anti-CD38 antibodies can be used. An exemplary anti-CD38 antibody is daratumumab (see, e.g., U.S. Pat. No. 7,829,673).

Another immune checkpoint protein that can be targeted in the methods provided herein is ICOS, also known as CD278. The complete protein sequence of human ICOS has Genbank accession number NP_036224. In some embodiments, the immune checkpoint inhibitor is an anti-ICOS antibody (e.g., a human antibody, a humanized antibody, or a chimeric antibody), an antigen-binding fragment thereof, an immunoadhesin, a fusion protein, or oligopeptide. Anti-human-ICOS antibodies (or VH and/or VL domains derived therefrom) suitable for use in the present methods can be generated using methods well known in the art. Alternatively, art-recognized anti-ICOS antibodies can be used. Exemplary anti-ICOS antibodies include JTX-2011 (see, e.g., WO 2016/154177, WO 2018/187191) and GSK3359609 (see, e.g., WO 2016/059602).

Another immune checkpoint protein that can be targeted in the methods provided herein is T-cell immunoreceptor with Ig and ITIM domains (TIGIT). The complete protein sequence of human TIGIT has Genbank accession number NP_776160. In some embodiments, the immune checkpoint inhibitor is an anti-TIGIT antibody (e.g., a human antibody, a humanized antibody, or a chimeric antibody), an antigen-binding fragment thereof, an immunoadhesin, a fusion protein, or oligopeptide. Anti-human-TIGIT antibodies (or VH and/or VL domains derived therefrom) suitable for use in the present methods can be generated using methods well known in the art. Alternatively, art-recognized anti-TIGIT antibodies can be used. An exemplary anti-TIGIT antibody is MK-7684 (see, e.g., WO 2017/030823, WO 2016/028656).

Another immune checkpoint protein that can be targeted in the methods provided herein is OX40, also known as CD134. The complete protein sequence of human OX40 has Genbank accession number NP_003318. In some embodiments, the immune checkpoint inhibitor is an anti-OX40 antibody (e.g., a human antibody, a humanized antibody, or a chimeric antibody), an antigen-binding fragment thereof, an immunoadhesin, a fusion protein, or oligopeptide. Anti-human-OX40 antibodies (or VH and/or VL domains derived therefrom) suitable for use in the present methods can be generated using methods well known in the art. Alternatively, art-recognized anti-OX40 antibodies can be used. An exemplary anti-OX40 antibody is PF-04518600 (see, e.g., WO 2017/130076). ATOR-1015 is a bispecific antibody targeting CTLA4 and OX40 (see, e.g., WO 2017/182672, WO 2018/091740, WO 2018/202649, WO 2018/002339).

Another immune checkpoint protein that can be targeted in the methods provided herein is glucocorticoid-induced tumor necrosis factor receptor-related protein (GITR), also known as TNFRSF18 and AITR. The complete protein sequence of human GITR has Genbank accession number NP_004186. In some embodiments, the immune checkpoint inhibitor is an anti-GITR antibody (e.g., a human antibody, a humanized antibody, or a chimeric antibody), an antigen-binding fragment thereof, an immunoadhesin, a fusion protein, or oligopeptide. Anti-human-GITR antibodies (or VH and/or VL domains derived therefrom) suitable for use in the present methods can be generated using methods well known in the art. Alternatively, art-recognized anti-GITR antibodies can be used. An exemplary anti-GITR antibody is TRX518 (see, e.g., WO 2006/105021).

Another immune checkpoint protein that can be targeted in the methods provided herein is T-cell immunoglobulin and mucin-domain containing-3 (TIM3), also known as HAVCR2. The complete protein sequence of human TIM3 has Genbank accession number NP_116171. In some embodiments, the immune checkpoint inhibitor is an anti-TIM3 antibody (e.g., a human antibody, a humanized antibody, or a chimeric antibody), an antigen-binding fragment thereof, an immunoadhesin, a fusion protein, or oligopeptide. Anti-human-TIM3 antibodies (or VH and/or VL domains derived therefrom) suitable for use in the present methods can be generated using methods well known in the art. Alternatively, art-recognized anti-TIM3 antibodies can be used. Exemplary anti-TIM3 antibodies include LY3321367 (see, e.g., WO 2018/039020), MBG453 (see, e.g., WO 2015/117002) and TSR-022 (see, e.g., WO 2018/085469).

Another immune checkpoint protein that can be targeted in the methods provided herein is 4-1BB, also known as CD137, TNFRSF9, and ILA. The complete protein sequence of human 4-1BB has Genbank accession number NP_001552. In some embodiments, the immune checkpoint inhibitor is an anti-4-1BB antibody (e.g., a human antibody, a humanized antibody, or a chimeric antibody), an antigen-binding fragment thereof, an immunoadhesin, a fusion protein, or oligopeptide. Anti-human-4-1BB antibodies (or VH and/or VL domains derived therefrom) suitable for use in the present methods can be generated using methods well known in the art. Alternatively, art-recognized anti-4-1BB antibodies can be used. An exemplary anti-4-1BB antibody is PF-05082566 (utomilumab; see, e.g., WO 2012/032433).

In some embodiment, the immune therapy could be adoptive immunotherapy, which involves the transfer of autologous antigen-specific T-cells generated ex vivo. The T-cells used for adoptive immunotherapy can be generated either by expansion of antigen-specific T-cells or redirection of T-cells through genetic engineering. Isolation and transfer of tumor-specific T cells has been shown to be successful in treating melanoma. Novel specificities in T-cells have been successfully generated through the genetic transfer of transgenic T-cell receptors or chimeric antigen receptors (CARs). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. CARs have successfully allowed T-cells to be redirected against antigens expressed at the surface of tumor cells from various malignancies including lymphomas and solid tumors.

In one embodiment, the present application provides for a combination therapy for the treatment of cancer wherein the combination therapy comprises adoptive T-cell therapy and a checkpoint inhibitor. In one aspect, the adoptive T-cell therapy comprises autologous and/or allogeneic T-cells. In another aspect, the autologous and/or allogeneic T-cells are targeted against tumor antigens. The engineered hSDH enzyme may be administered to the patient prior to and/or simultaneously with the administration of the adoptive T-cell therapy. In another aspect, the autologous and/or allogeneic T-cells may be engineered to express the engineered hSDH enzyme.

### C. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic composition and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

### D. Gene Therapy

In yet another embodiment, the secondary treatment is a gene therapy in which a therapeutic polynucleotide is administered before, after, or at the same time as the therapeutic composition. Viral vectors for the expression of a gene product are well known in the art, and include such eukaryotic expression systems as adenoviruses, adeno-associated viruses, retroviruses, herpesviruses, lentiviruses, poxviruses including vaccinia viruses, and papiloma viruses, including SV40. Alternatively, the administration of expression constructs can be accomplished with lipid based vectors such as liposomes or DOTAP: cholesterol vesicles.

### E. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatments provided herein, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present embodiments may be used in conjunction with removal of superficial cancers, precancers, or incidental amounts of normal tissue. In some aspects, following tumor resection a dendritic cell composition of the embodiments is administered to lymphoid tissue that drained the previous site for the tumor.

### V. Kits

Some embodiments concern kits, such as diagnostic and therapeutic kits, as well as kits for preparing and/or delivering exosomes or MVs. For example, a kit may comprise one or more pharmaceutical compositions as described herein and optionally instructions for their use. Kits may also comprise one or more devices for accomplishing administration of such compositions. For example, a subject kit may comprise a pharmaceutical composition and catheter for accomplishing direct administration of the composition to a patient having or at risk for a demyelination disorder. In other embodiments, a subject kit may comprise pre-filled ampoules of isolated exosomes, optionally formulated as a pharmaceutical, or lyophilized, for use with a delivery device. The technology herein also includes kits for generating MV from dendritic cells.

Kits may comprise a container with a label. Suitable containers include, for example, bottles, vials, and test tubes. The containers may be formed from a variety of materials such as glass or plastic. The container may hold a composition which includes an antibody that is effective for therapeutic or non-therapeutic applications, such as described above. The label on the container may indicate that the composition is used for a specific therapy or non-therapeutic application, and may also indicate directions for either in vivo or in vitro use, such as those described above. In some embodiments, kits will comprise the container described above and one or more other containers comprising materials desirable from a commercial and user standpoint, including buffers, diluents, filters, needles, syringes, and package inserts with instructions for use.

### VI. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

In sum, the data contained herein demonstrate that DC express and secrete both CTLA-4⁺ and CTLA-4^{neg} extracellular vesicles. T_{H}1 polarized DC secrete a higher ratio of CTLA-4^{neg} EV to CTLA-4⁺ EV, and T_{H}2 polarized DC secrete a lower ratio of CTLA-4^{neg} EV to CTLA-4⁺ EV; however, both types of EV may be recovered irrespective of DC T_{H} polarization status and appear to possess similar functional qualities, again irrespective of DC polarization status. CTLA-4^{neg} EV possess the ability to skew T cells toward a T_{H}1 polarized phenotype, inducing upregulated IFN-γ, granzyme B, CD25, and CD161 expression and a higher CD8 to CD4 ratio among proliferating T-cell blasts. CTLA-4⁺ EV impart the opposite effect, inducing downregulation of T_{H}1 polarizing and activation markers and upregulation of exhaustion markers such as PD-1 and Tim3. The data indicate that CTLA-4⁺ EV or “dexosomes” could be administered contextually in a manner useful for ameliorating autoimmune conditions, solid organ transplant rejection, or GVHD following HSCT. In contrast, CTLA-4^{neg} EV/dexosomes could be administered in contexts requiring enhancement of the T_{H}1 immune response including chronic viral infection or cancer immunotherapy.

### Example 2

Dendritic cells (DCs) are critical mediators of immunity which bridge the gap between the innate and adaptive arms of the immune response. They not only participate in frontline (innate) defenses against pathogens and tumors, they also drive subsequent adaptive immune responses and dictate the strength and timbre of such responses. The manner in which dendritic cells help tailor adaptive immune responses to the ongoing assault is an area of intense investigation, as these mechanisms involve a conglomeration of external and internal signals which drive DC differentiation and maturation pathways, specifically with regard to the surface receptors and soluble inflammatory mediators expressed by DCs (Cella et al., 1999; Sallusto et al., 1995; Sallusto et al., 1994). Additionally, DCs release extracellular vesicles (EVs) which themselves are reported to influence the immune response in a variety of ways (Segura et al., 2005; Thery et al., 2002; Wakim et al., 2011).

As reviewed elsewhere (Thery et al., 2009), extracellular vesicles (EV) are 30-1000 nm cargo-bearing, membrane-bound entities released from cells at the steady state and upon stimulation. These vesicles may be classified based on size, mode of biogenesis, ultracentrifugation properties and contents (Thery et al., 2009). In the case of exosomes (30-150 nm), parent cell proteins and nucleic acids (predominantly miRNA) are targeted to the endosomal membrane which then invaginates to enclose the targeted cargo, resulting in the formation of multivesicular bodies (MVB) wherein vesicles are present within an endosome. These vesicles are released to the extracellular space once fusion of the MVB membrane and the plasma membrane occurs (Babst et al., 2002; Odorizzi et al., 1998; Saksena et al., 2007; Stuffers et al., 2009). Alternatively, outward budding of the plasma membrane gives rise to microvesicles (100-1000 nm), ectosomes (50-200 nm) and apoptotic bodies (50-500 nm) which may be differentiated based on size, cargo and biogenesis (Thery et al., 2009; Heinen et al., 1999). DC-derived EVs are critical mediators of immune responses through the transfer of antigens between DCs and through their ability to prime immune responses independently of the parental DC (Segura et al., 2005; Thery et al., 2002; Wakin et al., 2011). These immune-modulatory effects as well as other EV-peculiar properties render DC-derived EVs (dexosomes) a favorable option for off-the-shelf cell-based immunotherapy. Specifically, EVs are sub-cellular entities that have no known direct cytolytic capabilities, as such the risk of graft-versus-host disease is limited. Additionally, the generation of sufficient DC numbers for cell-based vaccinations can be technically challenging and chemotherapies involved in the mobilization of these cells negatively impact patients' quality of life, dexosomes present an alternative modality through which antigen-specific responses may be generated from relatively fewer DCs. Finally, since dexosomes are subcellular entities, they may be impervious to the immunosuppressive tumor microenvironment, which often hampers cell-based vaccines.

In the inventor's attempts to harness the therapeutic potential of dexosomes, the inventor's past (Halpert et al., 2016) and current work demonstrated that the immunostimulatory effect of dexosomes were subdued by the presence of the immunosuppressive molecule, CTLA-4. The inventors previously determined that CTLA-4 expression was upregulated in mature DCs and subsequently packaged into dexosomes, which are released to the extracellular space. Uptake of these dexosomes by bystander DCs resulted in the reduction of surface B7 on the bystander, in a manner that was dependent on the CTLA-4 receptors CD80 and CD86. Further, co-culture of CTLA-4 siRNA-treated DCs with autologous T cells resulted in a significantly increased proportion of CD8⁺ CD25⁺ IFNy⁺ T cells, with a concurrent decrease in the Treg frequencies (CD4⁺ CD25⁺ FoxP3⁺) when compared to non-targeting siRNA-treated DCs (NT-siRNA). In a B16 mouse melanoma model, antitumor immunity and survival were significantly heightened among recipients of a CTLA-4 siRNA-treated DC vaccine, relative to NT-siRNA-treated DC vaccine recipients. These lines of evidence pointed to CTLA-4 and, in effect, dexosomes as key mediators of DC-driven CD8 T-cell responses in vitro and in vivo (Halpert et al., 2016).

Here, the inventors extend these previous findings by investigating the direct influence of CTLA-4⁺ dexosomes on T-cell effector functions, particularly in the generation of productive Type-1 (T1) immunity. Due to the critical importance of the T cell-DC synapse and hence proximity of DC to T cells for the generation of a robust immune response, the inventors hypothesized that CTLA-4⁺ dexosomes could directly moderate T1 responses in order to limit the generation of aberrant tissue-destructive programs. Through the inventor's human monocyte-derived dendritic cell models, conditional knockout mouse (cKO) model (CTLA-4^{fl/fl} CD11c^{Cre}) and mouse tumor models, the inventors demonstrate that CTLA-4⁺ dexosomes limit the generation of robust T1 immune responses, with implications for the design of DC-based immunotherapies as well as the T1 response-associated pathologies.

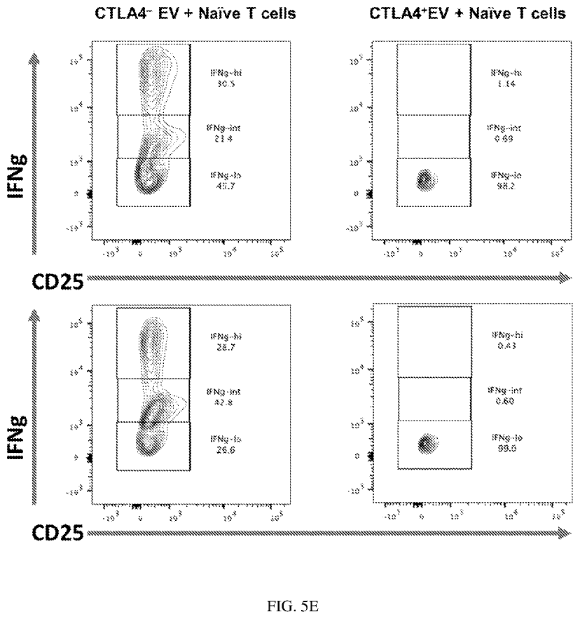

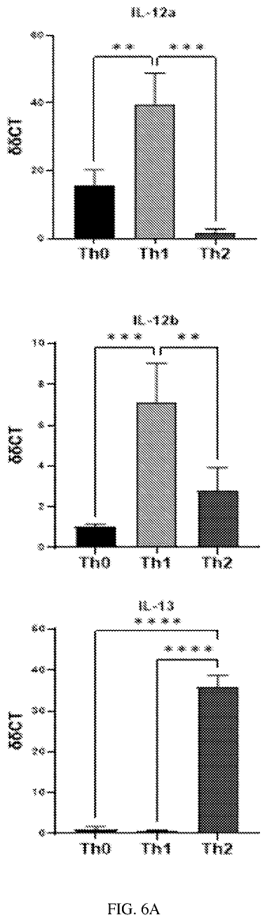

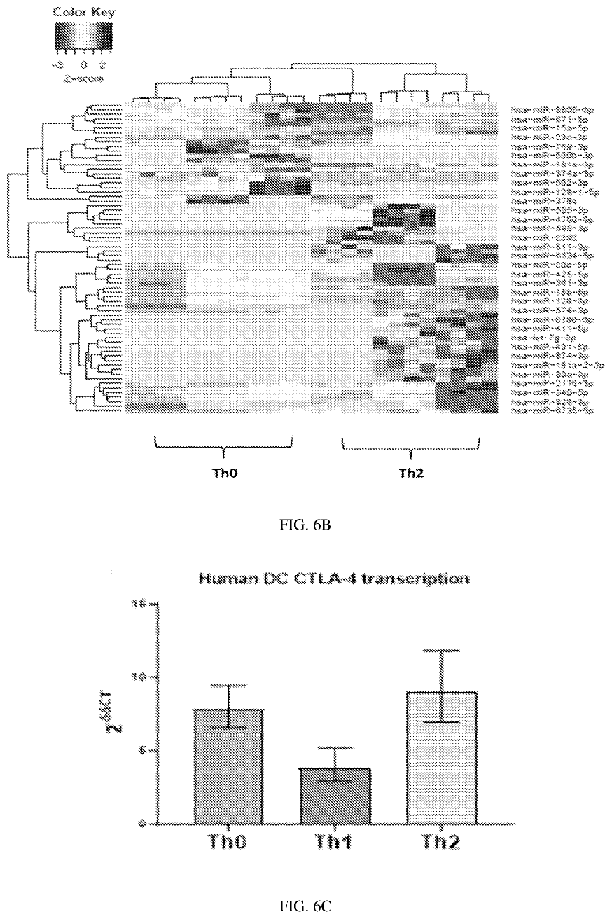

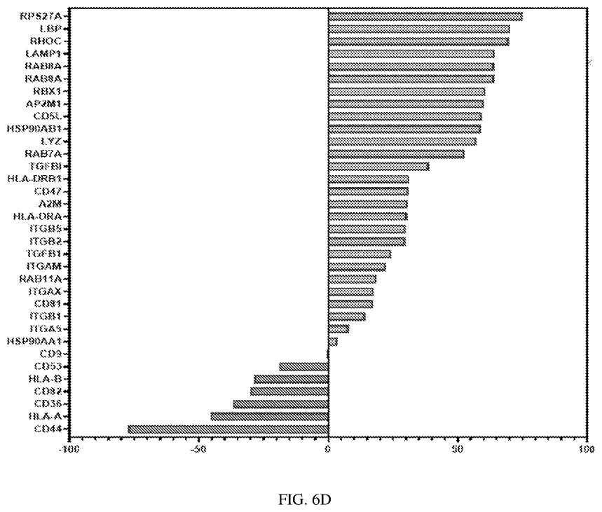

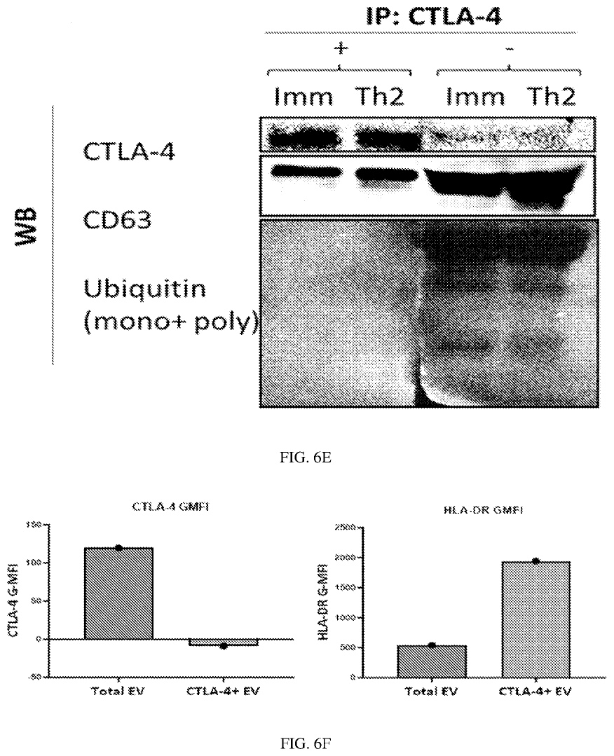

DC CTLA-4 levels are regulated by in vitro maturation conditions. At the steady state, the majority of human DC are resident in tissues with relatively small numbers detectable in peripheral blood. As such, harvesting tissue-resident DC for therapeutic as well as research purposes is often only possible in disease states wherein tissue biopsies, bone marrow, or whole organ collection is indicated. As such, the inventors' efforts to define the biology of this elusive leukocyte population in humans have predominantly relied on the in vitro generation of DC from monocytes (MoDC) isolated from peripheral blood. For the full therapeutic capabilities of DC to be actualized, past limitations to therapeutic success must be addressed by identifying prior roadblocks as well as ways in which they may be circumvented (Laureano et al., 2022). To this end, the inventor's previous work identified in vitro conditions by which moDC may be polarized towards a Th1 or Th2 response-promoting phenotype. These moDC may also be matured in a manner that drove T-cell responses without preference for the Th1 or Th2 response (hereafter referred to as Th0 DC). Within this paradigm, the inventors determined that Th1 DC are marked by heightened expression of IL12A and IL12B, while Th2 DC express little to no IL12A, low levels of IL12B but significantly elevated levels of IL13 and CTLA4 (FIGS. 6A and 6C). On the other hand, Th0 DC express intermediate levels of IL12A, IL12B and CTLA4 with little to no IL13.

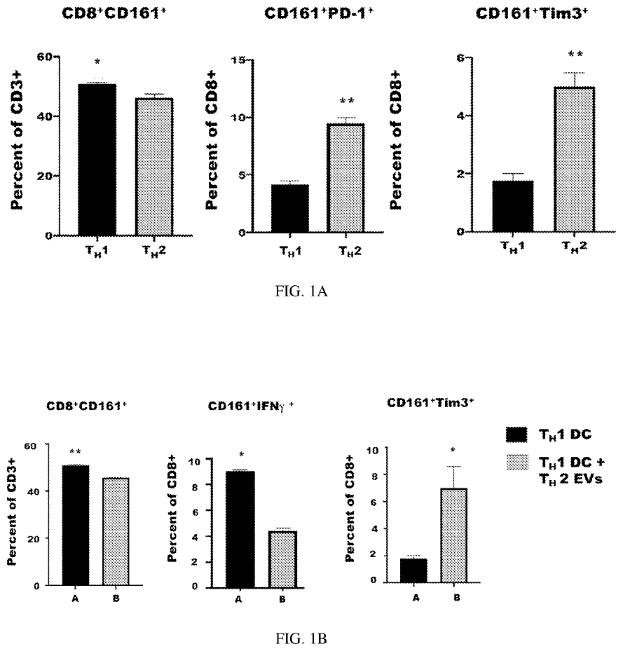

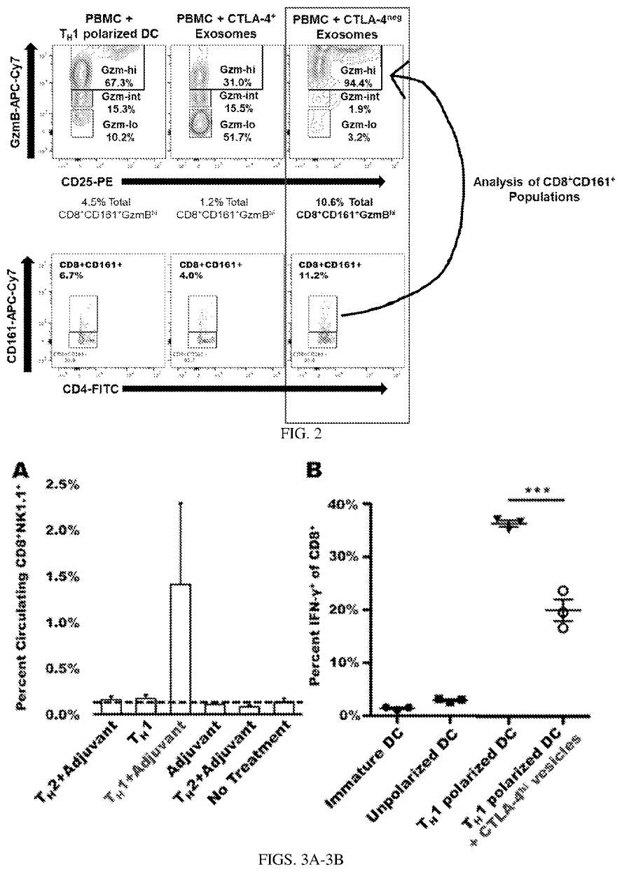

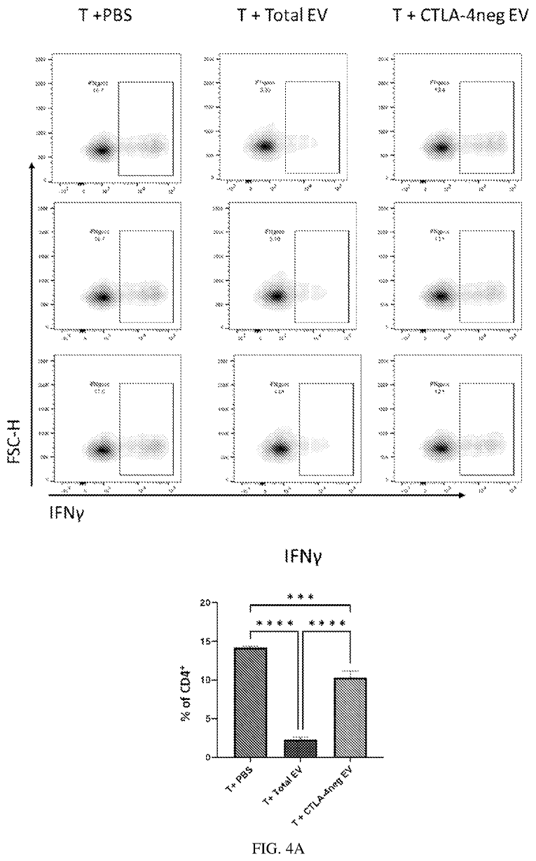

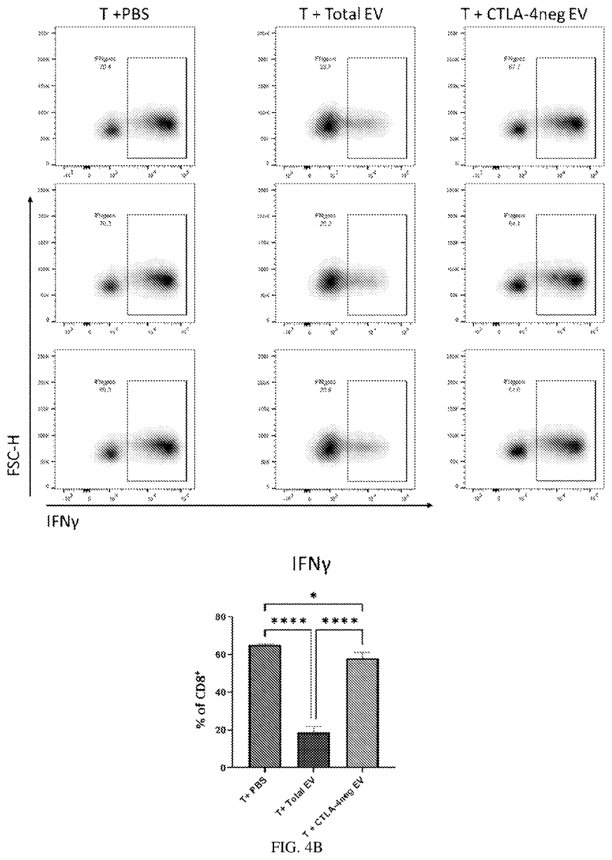

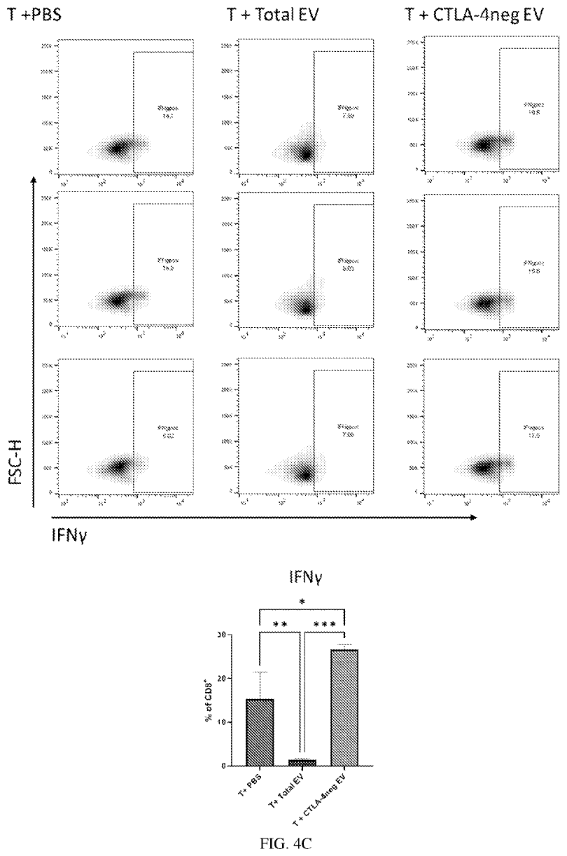

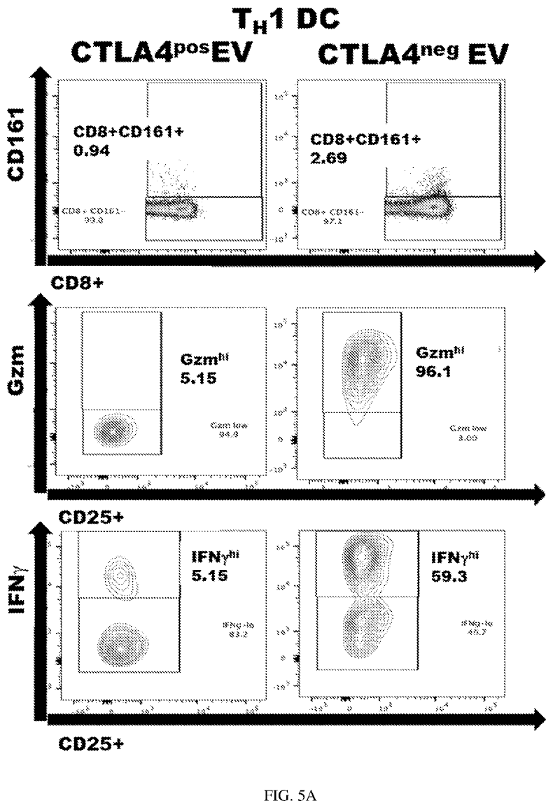

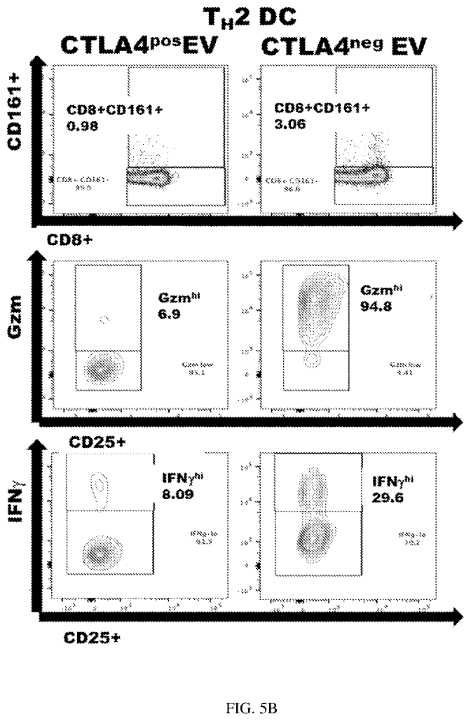

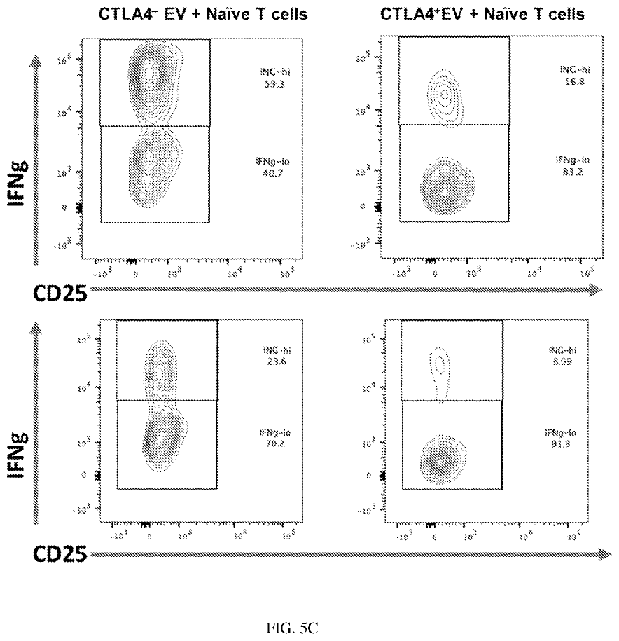

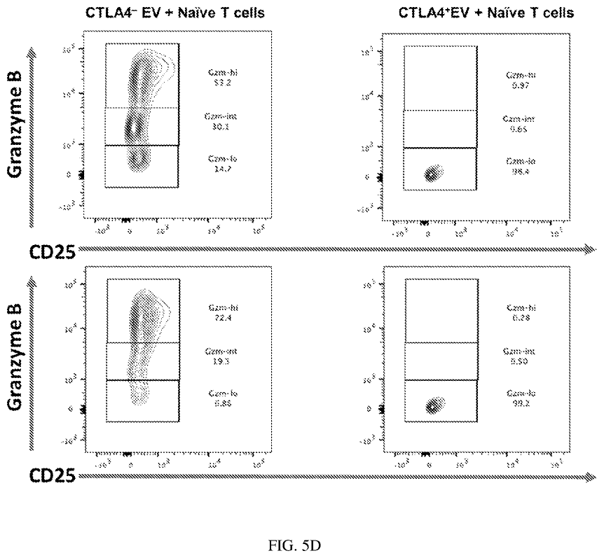

Of particular interest is the role of moDC-derived dexosomes in this Th-polarizing paradigm since their immunomodulatory effects are well-recorded although incompletely defined (Segura et al., 2005; Thery et al., 2002; Wakim et al., 2011). As such, the inventors extended their previous findings through miRNASeq analysis of Th0 and Th2 dexosomes to identify differences in miRNA cargo within these dexosomes that might provide insight into their differential effects on T cell responses. Through the analysis of vesicles from 3 independent donors, the inventors identified 69 miRNA species which were differentially represented within Th0 dexosomes in comparison to Th2 dexosomes (FIG. 6B). The protein cargo borne by dexosomes is another key component to defining the role of dexosomes in differential Th polarization. Accordingly, the inventors previously deduced that dexosomes of moDC contain a CTLA-4⁺ subset, the levels of which vary based on polarization state of the parental DC. To this end, the inventors immunoprecipitated CTLA-4⁺ dexosomes from immature or Th2 moDC bulk dexosome isolates and conducted western blot analyses. The inventors determined that although CTLA-4⁺ and CTLA-4^{neg} subsets both contained the extracellular vesicle marker, tetraspanin CD63, only the CTLA-4^{neg} subset was positive for ubiquitinated proteins (FIG. 1E). This lends insight into the differences in the biogenesis of these dexosome subsets. Ubiquitination has been shown to drive the packaging of proteins into multivesicular bodies (MVB) destined for release as extracellular vesicles through identification and translocation of such cargo by ESCRT proteins (Babst et al., 2002; Odorizzi et al., 1998; Saksena et al., 2007). Therefore, the inventors' findings demonstrate that proteins within the CTLA-4⁺ dexosome compartment are packaged independently of the ESCRT pathway. Next, the inventors sought to define the overall proteome of the CTLA-4⁺ and CTLA-4^{neg} compartment as a more efficient means to gain insight into the origins and effects of these dexosome subsets. Accordingly, the inventors conducted mass spectrometry analysis of the CTLA-4⁺ and CTLA-4^{neg} dexosomes from bulk Th2 dexosome isolates. Of note, the inventors observed enrichment of HLA Class II moieties, HSP90AB1, CD81, ITGAM (CD11b), and ITGAX (CD11c) in CTLA-4⁺ dexosomes but not of HLA Class I, CD82, CD36 (and CD5L, its soluble ligand) and CD44 in CTLA-4^{neg} dexosomes (FIG. 6D). Flow cytometry analysis of HLA Class II (HLA-DR) expression recapitulated this finding (FIG. 6F). Additionally, the inventors noted significant differences between the physical characteristics of CTLA-4⁺ and CTLA-4^{neg} EV fractions as determined by Cryo-Electron Microscopy. While the CTLA-4^{neg} fraction potentially constitutes a heterogeneous population of vesicles, the population was predominantly composed of vesicles of about 200 nm diameter or greater. These spherical vesicles were characterized by electron-dense membranes and lumen (FIG. 6H). The inventors also noted the presence of smaller vesicles which were encapsulated by larger ones, a phenomenon as has been described (Gallart-Palau et al., 2015; Milasan et al., 2016). On the other hand, CTLA-4⁺ dexosomes were consistently spherical vesicles of about 30 nm that were significantly more electron-dense than their CTLA-4^{neg} counterparts. Overall, the inventors show that CTLA-4⁺ vesicles are a subset of dexosomes with distinct miRNA, proteomic and physical profiles. Furthermore, the output of CTLA-4⁺ vesicles within the dexosome milieu could be regulated by in vitro culture conditions.

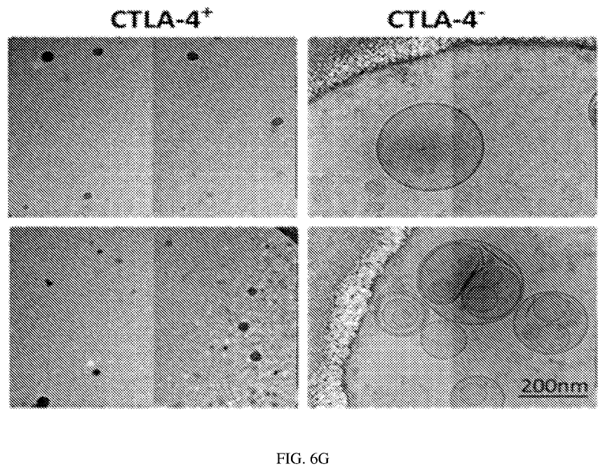

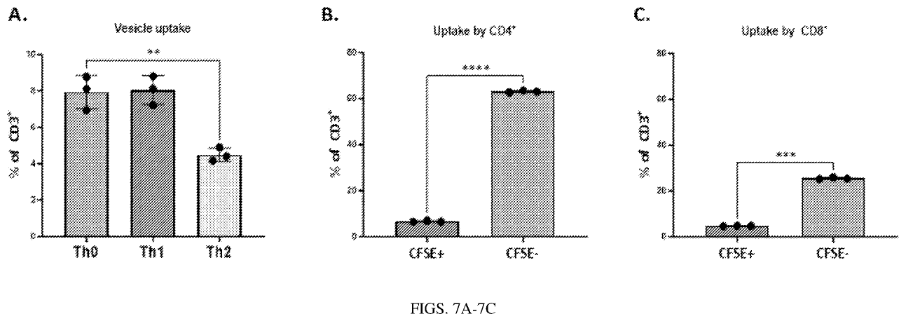

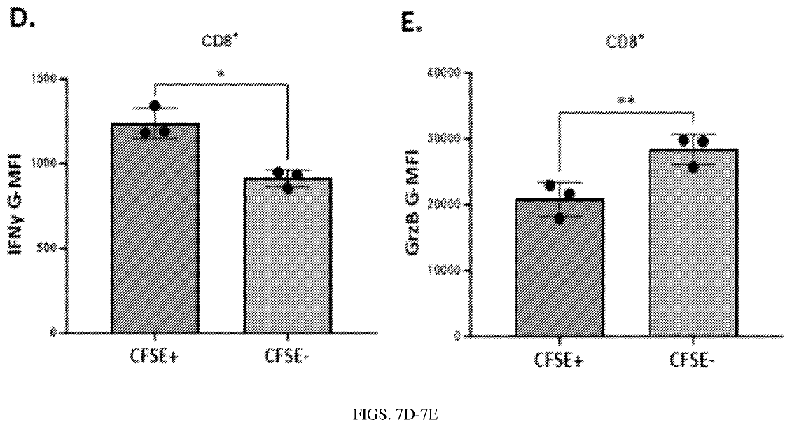

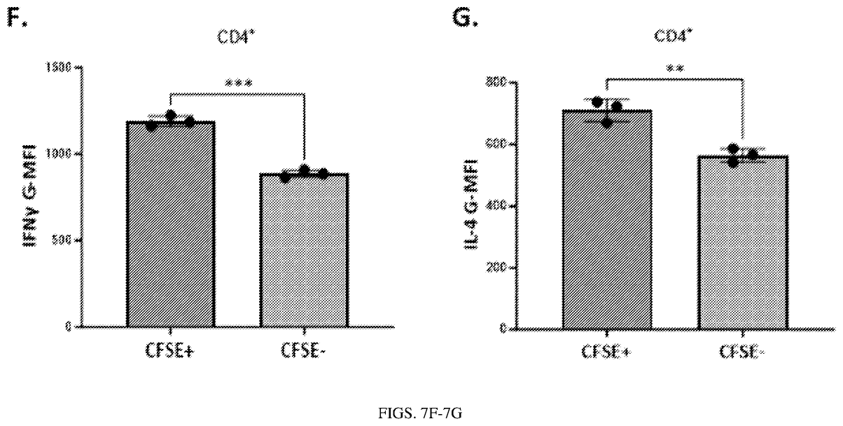

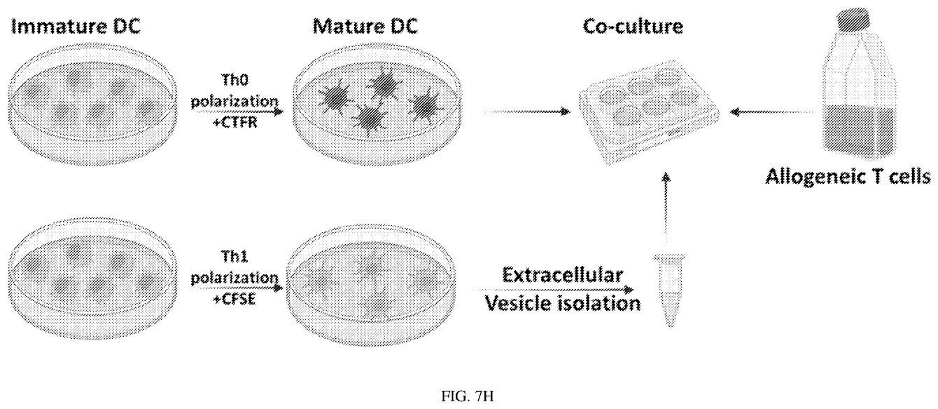

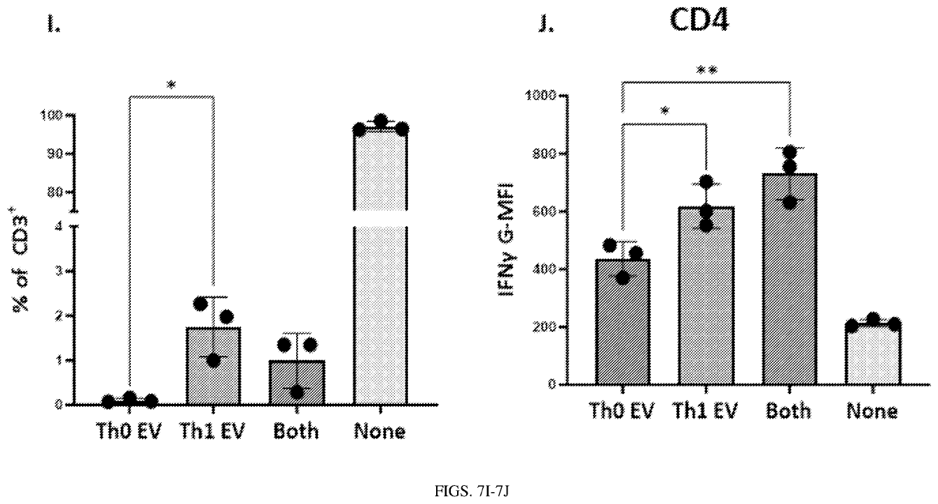

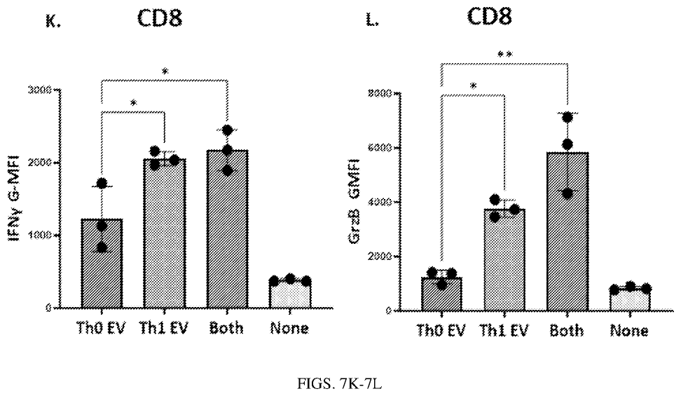

Parental DC and their dexosomes polarize T cell responses similarly. There is an increasing body of evidence which suggests that dexosomes are capable of driving T-cell responses. The understanding of mechanisms that govern this immunomodulatory effect has thus far been focused on the ability of dexosomes to suppress or enhance DC maturation (paracrine) as well as to cross-dress DC with the antigens that they bear. In this capacity, the effect of dexosomes on T cells is indirect and is predicated on the presence of other cells including B cells, NK cells and DC. There are additional lines of evidence that suggest that MHC-II⁺ and ICAM-1⁺ dexosomes may interact with T cells via LFA-1 on T cells leading to productive T-T antigen presentation. The effects of differentially polarized dexosomes on T-cell responses, however, remain to be defined. Further, the potency of dexosomes in the presence of DC with an opposing polarization state, as would be encountered upon in vivo therapeutic administration is unknown. To this end, the inventors first sought to determine whether there were differences in levels of differentially polarized dexosome uptake by T cells. The inventors matured and polarized DC in a Th0, Th1 or Th2 fashion and stained them with Carboxyfluorescein succinimidyl ester (CFSE) such that the dexosomes generated by these DC are marked by CFSE and uptake of these dexosomes by T cells can be tracked based on CFSE positivity. Mature, polarized DC were then cultured with unlabeled allogeneic T cells for 3 days, followed by flow cytometry analysis. The inventors observed similar levels of CFSE positivity (dexosome uptake) in T cells that had been cultured with Th0 and Th1 dexosomes (FIG. 7A). On the other hand, those cultured with Th2 dexosomes displayed significantly lower levels of vesicle uptake (FIG. 7A). Next, the inventors sought to determine whether the uptake of dexosomes resulted in changes in T-cell phenotype, specifically in the expression of effector molecules. Thus, the inventors cultured T cells with Th0-polarized CFSE-labeled DC for 3 days followed by flow cytometry analysis. Leveraging the Th-agnostic DC (Th0) in this manner permitted the concurrent evaluation of Th1 and Th2 effector molecules in relation to dexosome uptake status. The inventors observed similar levels of dexosome uptake within the CD4⁺ and CD8⁺ T-cell compartments (FIGS. 7B,7C). CFSE⁺ CD8⁺ T cells expressed significantly higher levels of IFNγ but lower levels of Granzyme B (FIGS. 7D,7E). CFSE⁺ CD4⁺ T cells expressed greater amounts of IFNγ and IL-4, recapitulating the Th-agnostic nature of Th0 DC (FIGS. 7F,7G). Finally, the inventors investigated whether dexosomes could impact T-cell responses independently of the parental DC, particularly when non-parental DC were present. Thus, the inventors cultured allogeneic T cells with CFSE labeled Th1 dexosomes and CellTrace Far Red (CTFR) labeled Th0 DC (FIG. 7H). In line with the previous results, the majority of T cells took up no dexosomes. However, among those that took up any dexosomes, those that took up Th1 dexosomes were present at a greater frequency than those that took up Th0 dexosomes or both Th1 and Th0 dexosomes (FIG. 7I). The expression of GrzB by CD8⁺T cells and IFNγ by CD4⁺ and CD8⁺ T cells was significantly greater in Th1 dexosome⁺ T cells (FIGS. 7J-7L). Interestingly, the uptake of both Th0 and Th1 dexosomes was additive with regard to the expression of these cytokines. In sum, Th1 dexosomes alone can advance the immunostimulatory phenotype of their parental DC despite the presence of non-parental DC.

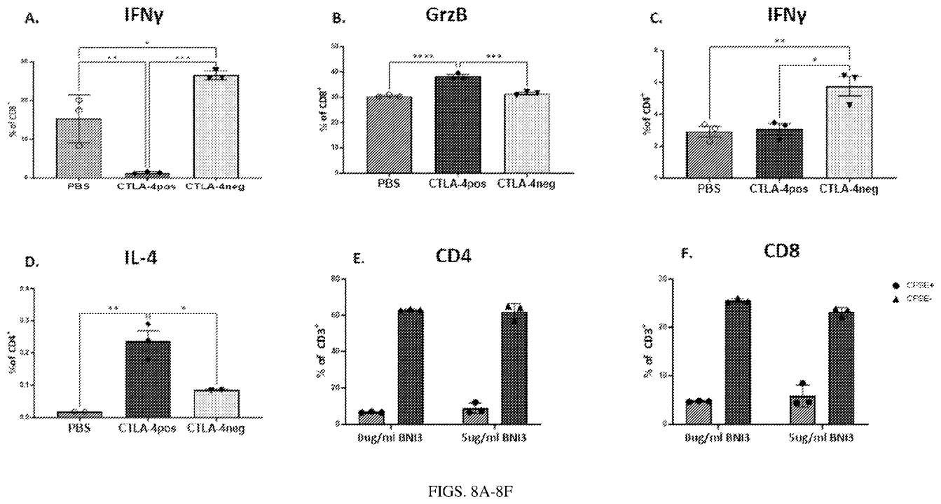

CTLA-4⁺ delineates dexosomes which inhibit Type-1 immune responses. Through the inventors' previous efforts to define the differences between Th1 and Th2 moDC, one key difference observed was in the expression of extracellular vesicle-borne CTLA-4 between these two DC types (Halpert et al., 2016). Specifically, Th2 DC expressed significantly more CTLA-4 than Th1 DC. In this context, CTLA-4 was shown to mediate uptake of dexosomes by neighboring DC (paracrine), leading to reduced expression of the co-stimulatory B7 molecules. Notably, the loss of CTLA-4 in moDC limited CD8⁺ T cell activation and IFNγ expression upon co-culture with allogeneic T cells. Subsequent experiments determined that the loss of CTLA-4 in a BMDC vaccine significantly improved survival and tumor clearance in a mouse melanoma model. Given these observations and the known role of CTLA-4 as a critical mediator of T1 T-cell responses, the inventors sought to investigate the role of CTLA-4⁺ dexosomes in the T-cell response. To this end, the inventors isolated dexosomes from Th2-skewed moDC and then depleted the CTLA-4⁺ fraction therein using antibody-bound immunomagnetic beads. Allogeneic T cells were then cultured with either CTLA-4^{neg} depleted dexosomes or total, CTLA-4^{neg} replete dexosomes. CTLA-4^{neg} containing dexosomes significantly suppressed CD4⁺ and CD8⁺ T cell IFNγ expression (FIGS. 8A-8D) while CTLA-4^{neg} depleted dexosomes rescued the expression of IFNγ. Notably, IL-4 and Granzyme B expression were perturbed in the opposite direction. Specifically, CTLA-4⁺ dexosomes promoted IL-4 and Granzyme B expression by CD4⁺ and CD8⁺ T cells, respectively. Next, the inventors sought to determine whether CTLA-4 mediated the interaction between dexosomes and T cells, as has been observed for DCs. Hence, the inventors cultured CFSE-labeled DC with T cells in the presence of anti-CTLA-4 (clone: BNI3). No difference in the uptake of dexosomes by CD4⁺ or CD8⁺ T cells was observed in the presence or absence of anti-CTLA-4 (FIGS. 8E,8F). Overall, the CTLA-4⁺ dexosome fraction limited T1 T cell responses although CTLA-4 itself was not required for the interaction between T cells and dexosomes.

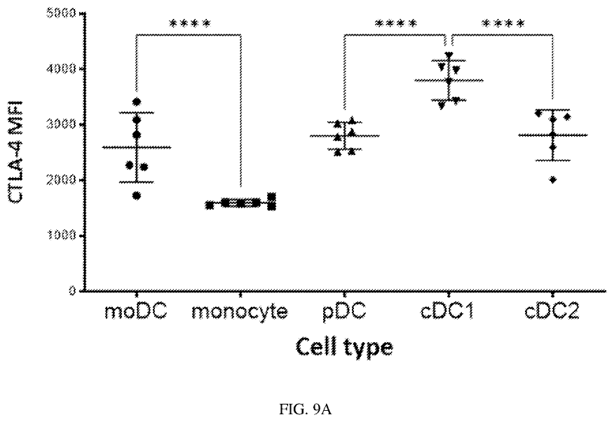

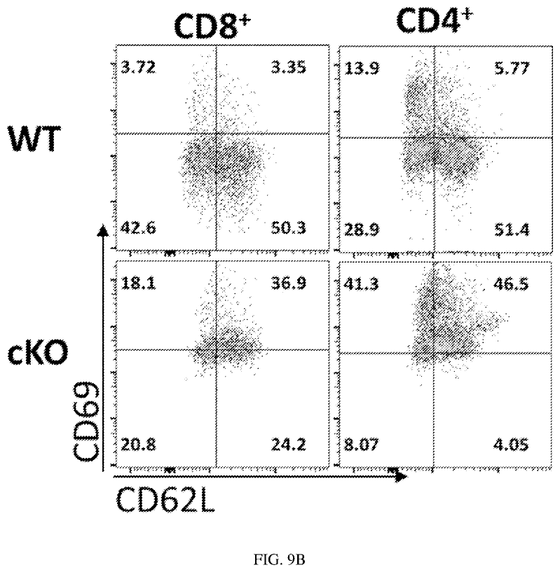

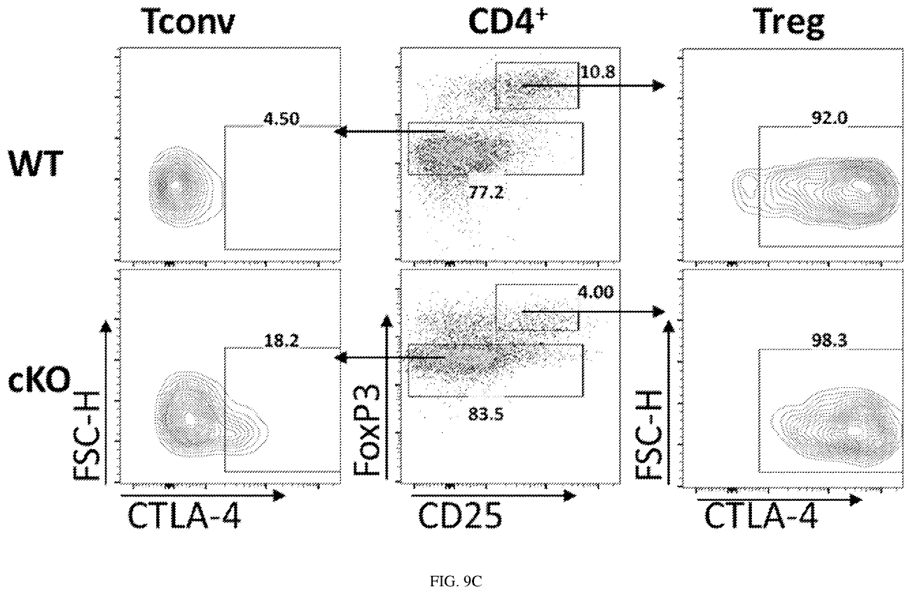

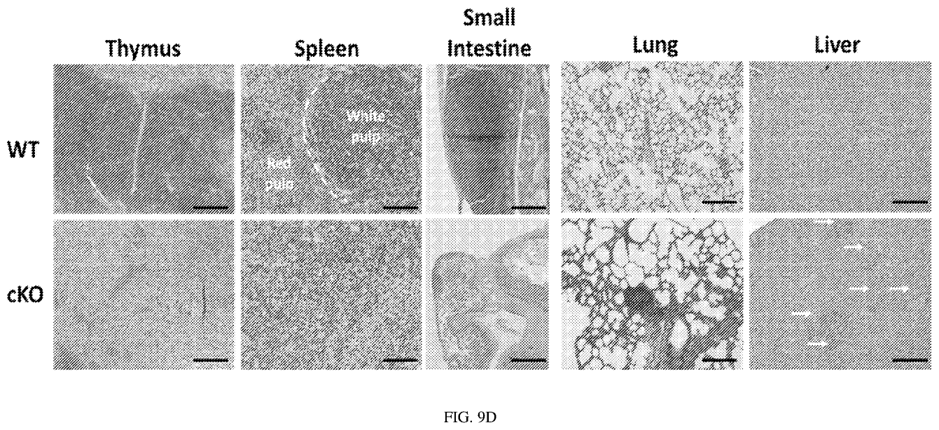

DC CTLA-4 is necessary for the maintenance of immune homeostasis. The DC compartment comprises moDC as well as plasmacytoid and conventional DC subsets in humans and in mice. Although the inventors' investigations have been focused on in vitro-differentiated moDC thus far, it is important to define the role of DC CTLA-4 in the in vivo setting, since these DC subsets play critical roles in the generation, maintenance, and cessation of immune responses. Accordingly, the inventors sought to define the CTLA-4 expression profile of the different DC subsets at the steady state. By flow cytometry analysis, the inventors observed the greatest levels of CTLA-4 expression in T1 conventional DC (cDC1). The expression of CTLA-4 was significantly lower and comparable between plasmacytoid DC (pDC) and type 2 conventional DC (cDC2) and moDC, while monocytes expressed the lowest levels of CTLA-4 (FIG. 9A). The inventors next generated conditional knockout mice (cKO) in which CD11c⁺ cells ceased to express CTLA-4 (CTLA-4^{fl/fl} CD11c^{Cre}) in order to define the role of CTLA-4 in the restraint of DC-mediated immune responses. In these mice, the loss of CTLA-4 resulted in immune dysregulation that was characterized by heightened levels of activated splenic T cells (FIGS. 9B,9C) and a reduction in the frequency of splenic Tregs. In comparison to wildtype mice, tissue architecture within the thymi, spleen, and Peyer's patches of cKO mice was severely disrupted in addition to significant reduction in the leucocyte occupancy within these tissues (FIG. 9D). Additionally, the inventors noted the absence of pancreatic tissue and a reduction in the number of Peyer's patches in cKO mice. In the lungs and livers of cKO mice, the inventors observed leukocytic inclusions as well as the enlargement of alveolar spaces in cKO mice. Ultimately, cKO mice were runted and succumbed to lethality within 6 weeks.

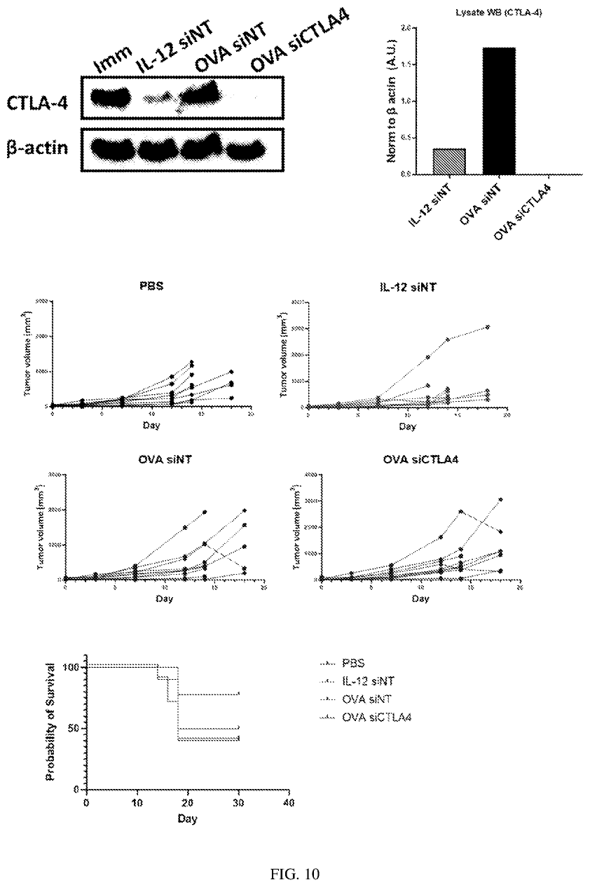

CTLA-4⁺ dexosomes limit anti-tumor immune responses in mice. Due to the differential T1 responses the inventors observed in the T cell-EV cultures, the inventors sought to investigate the role of these EV subsets in the generation of T1 responses in vivo. As such, the inventors employed a tumor model (B16-OVA) in which clearance and survival are predicated on productive T1 immunity. The inventors hypothesized that OVA-loaded, type-2 skewed EV could promote successful anti-tumor responses in the absence of CTLA-4, in line with in vitro observations. Accordingly, the inventors isolated EV from T1-skewed DC culture and from OVA protein-loaded type-2 response (T2) skewed DC which had been treated with siCTLA4 or non-targeting siRNA. B16-OVA-bearing mice were vaccinated with T1 EV, siCTLA4 T2 EV or siNT T2 EV when tumors became palpable and caliper measurements could be taken. Tumor growth, survival, lung metastases, and flow cytometry analysis on tumor-infiltrating lymphocytes were conducted in order to define the role of each of these EV types on the anti-tumor immune response (FIG. 10).

While promising in preclinical models, DC-derived EV (dexosome) Phase I and II trials for melanoma and NSCLC have failed to meet expectations (Escudier et al., 2005; Munich et al., 2012; Besse et al., 2016; Pujol et al., 2015). Therefore, defining the composition, biogenesis, and effects of dexosomes particularly as it relates to immunosuppressive CTLA-4 will be critical to leveraging their therapeutic potential. In the maintenance of tolerance to self and commensals as well as in mounting a robust adaptive immune response, dendritic cells play a crucial role. While the autoimmune-mediated lethality of CTLA-4^{-/-} mice underscores the importance of this molecule, it fails to define the role of immune-critical DCs in the biology of CTLA-4 and vice versa. Understanding the interplay between these two critical mediators of immunity will prove essential for defining important underlying mechanisms that mediate loss of tolerance, vaccine response as well as the suppression of anti-tumor immune responses.

Here, the inventors show that DC express CTLA-4 intracellularly and on the surface of extracellular vesicles (EV) which are released from DC. Additionally, the expression of CTLA-4 could be modulated by the culture conditions in which the parental DC were generated. Specifically, type-1 response-skewed DC (T1) expressed low levels of CTLA-4 and IL-13 in addition to high levels of IL-12. On the other hand, type-2-skewed DC (T2) expressed high levels of CTLA-4 and IL-13 along with low levels of IL-12. Co-culture of allogeneic T cells with bulk EV populations resulted in T cell phenotypes that recapitulated the effects of the parental DC.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Babst, M., Katzmann, D. J., Snyder, W. B., Wendland, B. & Emr, S. D. Endosome-associated complex, ESCRT-II, recruits transport machinery for protein sorting at the multivesicular body. Dev Cell 3, 283-289 (2002).
Besse, B. et al. Dendritic cell-derived exosomes as maintenance immunotherapy after first line chemotherapy in NSCLC. Oncoimmunology 5, e1071008 (2016).
Cella, M. et al. Maturation, activation, and protection of dendritic cells induced by double-stranded RNA. J Exp Med 189, 821-829 (1999).
Decker et al., Vaccine 24:3203-3216, 2006.
Escudier, B. et al. Vaccination of metastatic melanoma patients with autologous dendritic cell (DC) derived-exosomes: results of the first phase I clinical trial. J Transl Med 3, 10 (2005).
Gallart-Palau, X. et al. Extracellular vesicles are rapidly purified from human plasma by PRotein Organic Solvent PRecipitation (PROSPR). Sci Rep 5, 14664 (2015).
Halpert, M. M. et al. Dendritic Cell-Secreted Cytotoxic T-Lymphocyte-Associated Protein-4 Regulates the T-cell Response by Downmodulating Bystander Surface B7. Stem Cells Dev 25, 774-787 (2016).
Heijnen, H. F., Schiel, A. E., Fijnheer, R., Geuze, H. J. & Sixma, J. J. Activated platelets release two types of membrane vesicles: microvesicles by surface shedding and exosomes derived from exocytosis of multivesicular bodies and alpha-granules. Blood 94, 3791-3799 (1999).
Laureano, R. S. et al. Trial watch: Dendritic cell (DC)-based immunotherapy for cancer. Oncoimmunology 11, 2096363 (2022).
Milasan, A. et al. Extracellular vesicles are present in mouse lymph and their level differs in atherosclerosis. J Extracell Vesicles 5, 31427 (2016).
Munich, S., Sobo-Vujanovic, A., Buchser, W. J., Beer-Stolz, D. & Vujanovic, N. L. Dendritic cell exosomes directly kill tumor cells and activate natural killer cells via TNF superfamily ligands. Oncoimmunology 1, 1074-1083 (2012).
Odorizzi, G., Babst, M. & Emr, S. D. Fab1p PtdIns(3)P 5-kinase function essential for protein sorting in the multivesicular body. Cell 95, 847-858 (1998).
Pujol, J. L. et al. Safety and Immunogenicity of MAGE-A3 Cancer Immunotherapeutic with or without Adjuvant Chemotherapy in Patients with Resected Stage IB to III MAGE-A3-Positive Non-Small-Cell Lung Cancer. J Thorac Oncol 10, 1458-1467 (2015).
Saksena, S., Sun, J., Chu, T. & Emr, S. D. ESCRTing proteins in the endocytic pathway. Trends Biochem Sci 32, 561-573 (2007).
Sallusto, F. & Lanzavecchia, A. Efficient presentation of soluble antigen by cultured human dendritic cells is maintained by granulocyte/macrophage colony-stimulating factor plus interleukin 4 and downregulated by tumor necrosis factor alpha. J Exp Med 179, 1109-1118 (1994).
Sallusto, F., Cella, M., Danieli, C. & Lanzavecchia, A. Dendritic cells use macropinocytosis and the mannose receptor to concentrate macromolecules in the major histocompatibility complex class II compartment: downregulation by cytokines and bacterial products. J Exp Med 182, 389-400 (1995).
Segura, E. et al. ICAM-1 on exosomes from mature dendritic cells is critical for efficient naive T-cell priming. Blood 106, 216-223 (2005).
Stuffers, S., Sem Wegner, C., Stenmark, H. & Brech, A. Multivesicular endosome biogenesis in the absence of ESCRTs. Traffic 10, 925-937 (2009).
Thery, C. et al. Indirect activation of naive CD4+ T cells by dendritic cell-derived exosomes. Nat Immunol 3, 1156-1162 (2002).
Thery, C., Ostrowski, M. & Segura, E. Membrane vesicles as conveyors of immune responses. Nat Rev Immunol 9, 581-593 (2009).
Wakim, L. M. & Bevan, M. J. Cross-dressed dendritic cells drive memory CD8+ T-cell activation after viral infection. Nature 471, 629-632 (2011).

1. A method of generating extracellular vesicles (EV) from dendritic cells (DC), the method comprising (i) maturing DC towards either T_{H}1 polarization or T_{H}2 polarization, and (ii) isolating EV secreted by the polarized DC.

2. The method of claim 1, wherein maturing DC towards T_{H}1 polarization comprises maturing the DC in the presence of IL-12 or loading the DC with lysate and mRNA preparations from the same cell type.

3. The method of claim 1, wherein maturing DC towards T_{H}2 polarization comprises maturing the DC in the presence of S. aureus enterotoxin B (SEB).

4. The method of any one of claims 1-3, wherein the dendritic cells are monocytic dendritic cells.

5. The method of any one of claims 1-4, wherein the immature dendritic cells are monocyte-derived dendritic cells.

6. The method of claim 1, wherein the T_{H}1 polarized DC are IL-12⁺, IL-13^{lo}, and CTLA-4^{lo}.

7. The method of claim 1, wherein the T_{H}2 polarized DC are IL-13⁺, CTLA-4⁺, and IL-12^{lo}.

8. The method of any one of claims 1-7, further comprising separating the secreted EV into CTLA-4⁺ and CTLA-4^{neg} populations.

9. The method of claim 8, wherein the CTLA-4^{neg} EV population is obtained by depletion of CTLA-4⁺ EV.

10. The method of claim 8, wherein the CTLA-4⁺ EV population is obtained by positive selection using bead-bound anti-CTLA-4 antibodies.

11. A composition comprising dendritic cell-derived extracellular vesicles (EV) obtained by the method of any one of claims 1-10.

12. The composition of claim 11, wherein the EV are CTLA-4⁺.

13. The composition of claim 12, wherein the EV are derived from T_{H}2 polarized DC.

14. The composition of claim 12, wherein the EV are derived from T_{H}1 polarized DC.

15. The composition of claim 11, wherein the EV are CTLA-4^{neg}.

16. The composition of claim 15, wherein the EV are derived from T_{H}1 polarized DC.

17. The composition of claim 15, wherein the EV are derived from T_{H}2 polarized DC.

18. The composition of any one of claims 11-17, wherein the EV have a diameter of about 30-200 nm.

19. The composition of any one of claims 11-18, wherein the EV are CD63⁺, CD81⁺, and HLA-DR⁺.

20. A method of separating CTLA-4⁺ and CTLA-4^{neg} EV from each other to recover both populations of EV in a state that is physically and functionally intact, the method comprising isolating EV from mature DC cell culture supernatant using PEG precipitation and extracting the CTLA-4⁺ EV using bead-bound anti-CTLA-4 antibody, thereby separating CTLA-4⁺ and CTLA-4^{neg} EV from each other.

21. A method of inducing adaptive T_{H}1 polarization and activation of T cells, comprising contacting the T cells with CTLA-4^{neg} DC-derived EV.

22. The method of claim 21, wherein the T cells are CD8⁺ T cells.

23. The method of claim 21, wherein the T cells are CD4⁺ T cells.

24. The method of any one of claims 21-23, wherein the CTLA-4^{neg} DC-derived EV are according to any one of claims 15-17.

25. The method of any one of claims 21-24, wherein the T cells are present in bulk PBMC.

26. The method of any one of claims 21-24, wherein the T cells are isolated naïve T cells.

27. The method of any one of claims 21-24, wherein the T cells are in a subject, wherein the method comprises administering the EV to the subject.

28. A method of treating a disease in a patient in need thereof, the method comprising administering CTLA-4^{neg} DC-derived EV to the patient.

29. The method of claim 28, wherein the disease is a viral infection or a cancer.

30. The method of claim 28 or 29, wherein the DC-derived EV are administered intratumorally, intravenously, peritumorally, subcutaneously, and intraperitoneally.

31. The method of any one of claims 28-30, further comprising administering an immune checkpoint inhibitor to the patient.

32. The method of claim 31, wherein the immune checkpoint inhibitor is a CTLA-4 antagonist.

33. The method of claim 31 or 32, wherein the immune checkpoint inhibitor is ipilimumab, pembrolizumab or nivolumab.

34. A method of inhibiting adaptive T_{H}1 polarization and activation of CD8⁺ T cells, the method comprising contacting the T cells with CTLA-4⁺ DC-derived EV.

35. The method of claim 34, wherein the CTLA-4⁺ DC-derived EV are according to any one of claims 12-14.

36. The method of claim 34 or 35, wherein the T cells are present in bulk PBMC.

37. The method of claim 34 or 35, wherein the T cells are isolated naïve T cells.

38. The method of claim 34 or 35, wherein the T cells are in a subject, wherein the method comprises administering the EV to the subject.

39. The method of any one of claims 34-38, wherein the EVs are derived from T_{H}2 polarized DC.

40. A method of treating an autoimmune condition in a patient, the method comprising administering CTLA-4⁺ DC-derived EV to the patient.

41. The method of claim 40, wherein the CTLA-4⁺ DC-derived EV are according to any one of claims 12-14.

42. The method of claim 40 or 41, wherein the autoimmune condition is a T-cell mediated autoimmune condition.

43. The method of any one of claims 40-42, wherein the autoimmune condition is post-HSCT GVHD.

44. The method of any one of claims 40-43, wherein the CTLA-4⁺ DC-derived EV are administered intratumorally, intravenously, peritumorally, subcutaneously, and intraperitoneally.
